SEQUENCE LISTING (27 sequences)

SEQ ID NO: 1 - DNA, 3810 bp, Homo sapiens
  misc_feature 1..3810: note = Homo sapiens glial cell derived neurotrophic factor (GDNF)
  source 1..3810: mol_type = genomic DNA
ccgcctccagcgcgcccttgctgccccgcgcgaccccaggattgcgaactcttgcccctgacctgttgggcggggctccgcgctccagccatcagcccggatgggtctcctggctgggacttggggcacctggagttaatgtccaacctagggtctgcggagacccgatccgaggtgccgccgccggacgggactttaagatgaagttatgggatgtcgtggctgtctgcctggtgctgctccacaccgcgtccgccttcccgctgcccgccggtaagaggcctcccgaggcgcccgccgaagaccgctccctcggccgccgccgcgcgcccttcgcgctgagcagtgactcaaatatgccagaggattatcctgatcagttcgatgatgtcatggattttattcaagccaccattaaaagactgaaaaggtcaccagataaacaaatggcagtgcttcctagaagagagcggaatcggcaggctgcagctgccaacccagagaattccagaggaaaaggtcggagaggccagaggggcaaaaaccggggttgtgtcttaactgcaatacatttaaatgtcactgacttgggtctgggctatgaaaccaaggaggaactgatttttaggtactgcagcggctcttgcgatgcagctgagacaacgtacgacaaaatattgaaaaacttatccagaaatagaaggctggtgagtgacaaagtagggcaggcatgttgcagacccatcgcctttgatgatgacctgtcgtttttagatgataacctggtttaccatattctaagaaagcattccgctaaaaggtgtggatgtatctgactccggctccagagactgctgtgtattgcattcctgctacagtgcaaagaaagggaccaaggttcccaggaaatgtttgcccagaatggaagatgaggaccaaggaggcggaggaggaggaagaagaagaggaggaggaggaggaggaggaggaggaggaggaaggcagccatcatgggagcctggtagagggagatccagctacagacaactggacaggagagagagaaaacagccctctggattctccaggatggcagccgatgtcactagaagctcagggctgatgttcctggttggctattgccaccatttcagctgatacagtccaccatcactgattaccggcgcggttgcggtggatgcacttgaaccaaaccagtgtatctcctgtgatttgttttcatgtgtccgaagacaccagggaaacagagatcctggtgttgttccttgttattacgttttactgctgaaagtaagaggtttatttttctgtcactcagtggagacataccctggaaaggagaggggaaaaaaaaagcaaagatacaagagataatcacctttgcatttgaaagttgaggcccgaggtttactacaaccagcatttttgccaacggttggtgattgatttccatcacggtgtgtggggtgggaagaagttggctaggaaccaaaaaggctgtgctcatgattaaacacaaacctgaaggtatttcctttatgtccttggaaacaggaaacgagttgtggttttcgccagcattcttgtaggagagaatcggggaaggccccgaactgcccccgggcagggagagcccctcaggcctgttggtttacagagagacagatgttacataaccagctccgttgatgcgtggtcaccagtgaccagagaagctactcgatgcaatgcatctgtttcagatacagaaatatagagaagatatttattgaaatttaagttattgttatttattaccgttcactaatgaatttctcttttttcccttatttattaaagtttcttttcaaaggtgccaaagtatatgtgctcgcaaaatgcaaagaaaggtgacaaaaggaaatttgaattgggaacaagggtccatgcttttcaaagtattaaaaagttttttgccaggcaaaaatcacttactttacctttttaagaaaatttgtcattaattttccccagatttcagcatttttcccaatttttatttgtggagcatctcaggcaagccccctttcctggagcagcgtgcagagaccactggcacttgactttatttcttccttgctccattgctgaacagaaatgtcgtgggctccacttcctgttgtctttaagctcttagtcccctccacgtatacctatctgtactatgcataaccatatgtagaaaaggttcagttccttttagtaggtagtcctggatttaatgctgacctaaaagtaatgtcgacaatgctgtcaggtagctgccgttctaccgactccctccatccctgcccacccactgccctcccgagaatatgctggctgcccagtgcagcccgggagacacaggggccttccagaggtagggtctaccaggtcctgtacaacccctgggctgtcaccgggggtcaacagctgctgctcctatatacccaaacacctgacagctccctggggagcagatggctgagaagggtgctgaggaagccatattgggaccagccacagccacacacatggagcctcatacttaggagcgtgctgcctttaaatgaaggtggtcggggccagtgcagcggctcacacccataatcccaacactttggaaagccaaggtgggaggatctcttgaacccaggagtttgagaccagcttgggcaacatagggagaccctgtctctacagaaactttaaaaattaggcaggcatgatggtgcacacctgtggtcccagctactcaagaggctgaaggaggatcacttgagtccagaaggtcgaggctgcagtgagctgtgatcatgccactgcactccagcctaagtgacagtgcggtaccctgtctcaaaaaaaaaaaaaaaaaaaaaaagaggttggagcaggaggaagcataggggcgggaacagccacctcctccatgccctagattgtgaatttatcgggcagccaacacatgtatgacacactaggccctgtattacagcttgttacgcatttcataaaagggattttcattaaggagataatctattactacctaccttagtggctactagtataaaactatgacagatttagcaattaaatgaaatactggcctccatcaaataatcatagtaacaagaagcagcagttaccagacatctgatccccttcccccaaaatacccaaattcttcatggttctgcccttctctgtcctttctgctccccttgctcgcctgggaaatggaggaaaggccttccctctcacactgtcttgggatcttgctgagaattcagactgctcgaaacagtgacaaaccccagccatccagtcattcgtggagcacaatttggatgtggccccaggggcatctgtcccattcagagaaccttggcagtgcgatggccactgttcccaggcttcaacctcagtgaccccccccaacaactccccatggagagtccctgcccaaaaaagctgtaggatccaaggggtgtcaatagctcgttcccggcatcacctacacaccacaagcaggttttaatggaagcaagttgctccaccaaatccacaaaagggtaaagtttgtgatttttctttatcattgcgatcaccatctgataccgtaaggagtgcacttgtttggaagttctgacttctctgatctgtcttggtcgtttgtgttataaaaccaaagttctctacagactttatttttgtacaatatcattttgtaactttttacaaataaaaactcatttctattgc

SEQ ID NO: 2 - DNA, 3655 bp, Homo sapiens
  misc_feature 1..3655: note = Homo sapiens glial cell derived neurotrophic factor (GDNF)
  source 1..3655: mol_type = genomic DNA
catacaggccaaaagtctccaagtccctgctaacttcttgctctcgcaacagaatacctatttaggtgggaagaatgaggtgtgggcggcaggctgggtgccgccgccggacgggactttaagatgaagttatgggatgtcgtggctgtctgcctggtgctgctccacaccgcgtccgccttcccgctgcccgccgcaaatatgccagaggattatcctgatcagttcgatgatgtcatggattttattcaagccaccattaaaagactgaaaaggtcaccagataaacaaatggcagtgcttcctagaagagagcggaatcggcaggctgcagctgccaacccagagaattccagaggaaaaggtcggagaggccagaggggcaaaaaccggggttgtgtcttaactgcaatacatttaaatgtcactgacttgggtctgggctatgaaaccaaggaggaactgatttttaggtactgcagcggctcttgcgatgcagctgagacaacgtacgacaaaatattgaaaaacttatccagaaatagaaggctggtgagtgacaaagtagggcaggcatgttgcagacccatcgcctttgatgatgacctgtcgtttttagatgataacctggtttaccatattctaagaaagcattccgctaaaaggtgtggatgtatctgactccggctccagagactgctgtgtattgcattcctgctacagtgcaaagaaagggaccaaggttcccaggaaatgtttgcccagaatggaagatgaggaccaaggaggcggaggaggaggaagaagaagaggaggaggaggaggaggaggaggaggaggaggaaggcagccatcatgggagcctggtagagggagatccagctacagacaactggacaggagagagagaaaacagccctctggattctccaggatggcagccgatgtcactagaagctcagggctgatgttcctggttggctattgccaccatttcagctgatacagtccaccatcactgattaccggcgcggttgcggtggatgcacttgaaccaaaccagtgtatctcctgtgatttgttttcatgtgtccgaagacaccagggaaacagagatcctggtgttgttccttgttattacgttttactgctgaaagtaagaggtttatttttctgtcactcagtggagacataccctggaaaggagaggggaaaaaaaaagcaaagatacaagagataatcacctttgcatttgaaagttgaggcccgaggtttactacaaccagcatttttgccaacggttggtgattgatttccatcacggtgtgtggggtgggaagaagttggctaggaaccaaaaaggctgtgctcatgattaaacacaaacctgaaggtatttcctttatgtccttggaaacaggaaacgagttgtggttttcgccagcattcttgtaggagagaatcggggaaggccccgaactgcccccgggcagggagagcccctcaggcctgttggtttacagagagacagatgttacataaccagctccgttgatgcgtggtcaccagtgaccagagaagctactcgatgcaatgcatctgtttcagatacagaaatatagagaagatatttattgaaatttaagttattgttatttattaccgttcactaatgaatttctcttttttcccttatttattaaagtttcttttcaaaggtgccaaagtatatgtgctcgcaaaatgcaaagaaaggtgacaaaaggaaatttgaattgggaacaagggtccatgcttttcaaagtattaaaaagttttttgccaggcaaaaatcacttactttacctttttaagaaaatttgtcattaattttccccagatttcagcatttttcccaatttttatttgtggagcatctcaggcaagccccctttcctggagcagcgtgcagagaccactggcacttgactttatttcttccttgctccattgctgaacagaaatgtcgtgggctccacttcctgttgtctttaagctcttagtcccctccacgtatacctatctgtactatgcataaccatatgtagaaaaggttcagttccttttagtaggtagtcctggatttaatgctgacctaaaagtaatgtcgacaatgctgtcaggtagctgccgttctaccgactccctccatccctgcccacccactgccctcccgagaatatgctggctgcccagtgcagcccgggagacacaggggccttccagaggtagggtctaccaggtcctgtacaacccctgggctgtcaccgggggtcaacagctgctgctcctatatacccaaacacctgacagctccctggggagcagatggctgagaagggtgctgaggaagccatattgggaccagccacagccacacacatggagcctcatacttaggagcgtgctgcctttaaatgaaggtggtcggggccagtgcagcggctcacacccataatcccaacactttggaaagccaaggtgggaggatctcttgaacccaggagtttgagaccagcttgggcaacatagggagaccctgtctctacagaaactttaaaaattaggcaggcatgatggtgcacacctgtggtcccagctactcaagaggctgaaggaggatcacttgagtccagaaggtcgaggctgcagtgagctgtgatcatgccactgcactccagcctaagtgacagtgcggtaccctgtctcaaaaaaaaaaaaaaaaaaaaaaagaggttggagcaggaggaagcataggggcgggaacagccacctcctccatgccctagattgtgaatttatcgggcagccaacacatgtatgacacactaggccctgtattacagcttgttacgcatttcataaaagggattttcattaaggagataatctattactacctaccttagtggctactagtataaaactatgacagatttagcaattaaatgaaatactggcctccatcaaataatcatagtaacaagaagcagcagttaccagacatctgatccccttcccccaaaatacccaaattcttcatggttctgcccttctctgtcctttctgctccccttgctcgcctgggaaatggaggaaaggccttccctctcacactgtcttgggatcttgctgagaattcagactgctcgaaacagtgacaaaccccagccatccagtcattcgtggagcacaatttggatgtggccccaggggcatctgtcccattcagagaaccttggcagtgcgatggccactgttcccaggcttcaacctcagtgaccccccccaacaactccccatggagagtccctgcccaaaaaagctgtaggatccaaggggtgtcaatagctcgttcccggcatcacctacacaccacaagcaggttttaatggaagcaagttgctccaccaaatccacaaaagggtaaagtttgtgatttttctttatcattgcgatcaccatctgataccgtaaggagtgcacttgtttggaagttctgacttctctgatctgtcttggtcgtttgtgttataaaaccaaagttctctacagactttatttttgtacaatatcattttgtaactttttacaaataaaaactcatttctattgc

SEQ ID NO: 3 - DNA, 3830 bp, Homo sapiens
  misc_feature 1..3830: note = Homo sapiens glial cell derived neurotrophic factor (GDNF)
  source 1..3830: mol_type = genomic DNA
ccaaagcgtccgagactgggtacagtcgtccaggcgtgacgggggcgcggggagccagtgactcctctgggaggggaagggattagggccagaatctctcaaaggtgcaaaaatccagtcaagagagggttttcgggtataccacggaggattaaaactttcaagacaaatgcagtctttgcctaacagcaatggtgccgccgccggacgggactttaagatgaagttatgggatgtcgtggctgtctgcctggtgctgctccacaccgcgtccgccttcccgctgcccgccggtaagaggcctcccgaggcgcccgccgaagaccgctccctcggccgccgccgcgcgcccttcgcgctgagcagtgactcaaatatgccagaggattatcctgatcagttcgatgatgtcatggattttattcaagccaccattaaaagactgaaaaggtcaccagataaacaaatggcagtgcttcctagaagagagcggaatcggcaggctgcagctgccaacccagagaattccagaggaaaaggtcggagaggccagaggggcaaaaaccggggttgtgtcttaactgcaatacatttaaatgtcactgacttgggtctgggctatgaaaccaaggaggaactgatttttaggtactgcagcggctcttgcgatgcagctgagacaacgtacgacaaaatattgaaaaacttatccagaaatagaaggctggtgagtgacaaagtagggcaggcatgttgcagacccatcgcctttgatgatgacctgtcgtttttagatgataacctggtttaccatattctaagaaagcattccgctaaaaggtgtggatgtatctgactccggctccagagactgctgtgtattgcattcctgctacagtgcaaagaaagggaccaaggttcccaggaaatgtttgcccagaatggaagatgaggaccaaggaggcggaggaggaggaagaagaagaggaggaggaggaggaggaggaggaggaggaggaaggcagccatcatgggagcctggtagagggagatccagctacagacaactggacaggagagagagaaaacagccctctggattctccaggatggcagccgatgtcactagaagctcagggctgatgttcctggttggctattgccaccatttcagctgatacagtccaccatcactgattaccggcgcggttgcggtggatgcacttgaaccaaaccagtgtatctcctgtgatttgttttcatgtgtccgaagacaccagggaaacagagatcctggtgttgttccttgttattacgttttactgctgaaagtaagaggtttatttttctgtcactcagtggagacataccctggaaaggagaggggaaaaaaaaagcaaagatacaagagataatcacctttgcatttgaaagttgaggcccgaggtttactacaaccagcatttttgccaacggttggtgattgatttccatcacggtgtgtggggtgggaagaagttggctaggaaccaaaaaggctgtgctcatgattaaacacaaacctgaaggtatttcctttatgtccttggaaacaggaaacgagttgtggttttcgccagcattcttgtaggagagaatcggggaaggccccgaactgcccccgggcagggagagcccctcaggcctgttggtttacagagagacagatgttacataaccagctccgttgatgcgtggtcaccagtgaccagagaagctactcgatgcaatgcatctgtttcagatacagaaatatagagaagatatttattgaaatttaagttattgttatttattaccgttcactaatgaatttctcttttttcccttatttattaaagtttcttttcaaaggtgccaaagtatatgtgctcgcaaaatgcaaagaaaggtgacaaaaggaaatttgaattgggaacaagggtccatgcttttcaaagtattaaaaagttttttgccaggcaaaaatcacttactttacctttttaagaaaatttgtcattaattttccccagatttcagcatttttcccaatttttatttgtggagcatctcaggcaagccccctttcctggagcagcgtgcagagaccactggcacttgactttatttcttccttgctccattgctgaacagaaatgtcgtgggctccacttcctgttgtctttaagctcttagtcccctccacgtatacctatctgtactatgcataaccatatgtagaaaaggttcagttccttttagtaggtagtcctggatttaatgctgacctaaaagtaatgtcgacaatgctgtcaggtagctgccgttctaccgactccctccatccctgcccacccactgccctcccgagaatatgctggctgcccagtgcagcccgggagacacaggggccttccagaggtagggtctaccaggtcctgtacaacccctgggctgtcaccgggggtcaacagctgctgctcctatatacccaaacacctgacagctccctggggagcagatggctgagaagggtgctgaggaagccatattgggaccagccacagccacacacatggagcctcatacttaggagcgtgctgcctttaaatgaaggtggtcggggccagtgcagcggctcacacccataatcccaacactttggaaagccaaggtgggaggatctcttgaacccaggagtttgagaccagcttgggcaacatagggagaccctgtctctacagaaactttaaaaattaggcaggcatgatggtgcacacctgtggtcccagctactcaagaggctgaaggaggatcacttgagtccagaaggtcgaggctgcagtgagctgtgatcatgccactgcactccagcctaagtgacagtgcggtaccctgtctcaaaaaaaaaaaaaaaaaaaaaaagaggttggagcaggaggaagcataggggcgggaacagccacctcctccatgccctagattgtgaatttatcgggcagccaacacatgtatgacacactaggccctgtattacagcttgttacgcatttcataaaagggattttcattaaggagataatctattactacctaccttagtggctactagtataaaactatgacagatttagcaattaaatgaaatactggcctccatcaaataatcatagtaacaagaagcagcagttaccagacatctgatccccttcccccaaaatacccaaattcttcatggttctgcccttctctgtcctttctgctccccttgctcgcctgggaaatggaggaaaggccttccctctcacactgtcttgggatcttgctgagaattcagactgctcgaaacagtgacaaaccccagccatccagtcattcgtggagcacaatttggatgtggccccaggggcatctgtcccattcagagaaccttggcagtgcgatggccactgttcccaggcttcaacctcagtgaccccccccaacaactccccatggagagtccctgcccaaaaaagctgtaggatccaaggggtgtcaatagctcgttcccggcatcacctacacaccacaagcaggttttaatggaagcaagttgctccaccaaatccacaaaagggtaaagtttgtgatttttctttatcattgcgatcaccatctgataccgtaaggagtgcacttgtttggaagttctgacttctctgatctgtcttggtcgtttgtgttataaaaccaaagttctctacagactttatttttgtacaatatcattttgtaactttttacaaataaaaactcatttctattgc

SEQ ID NO: 4 - DNA, 3752 bp, Homo sapiens
  misc_feature 1..3752: note = Homo sapiens glial cell derived neurotrophic factor (GDNF)
  source 1..3752: mol_type = genomic DNA
ccaaagcgtccgagactgggtacagtcgtccaggcgtgacgggggcgcggggagccagtgactcctctgggaggggaagggattagggccagaatctctcaaaggtgcaaaaatccagtcaagagagggttttcgggtataccacggaggattaaaactttcaagacaaatgcagtctttgcctaacagcaatggtgccgccgccggacgggactttaagatgaagttatgggatgtcgtggctgtctgcctggtgctgctccacaccgcgtccgccttcccgctgcccgccgcaaatatgccagaggattatcctgatcagttcgatgatgtcatggattttattcaagccaccattaaaagactgaaaaggtcaccagataaacaaatggcagtgcttcctagaagagagcggaatcggcaggctgcagctgccaacccagagaattccagaggaaaaggtcggagaggccagaggggcaaaaaccggggttgtgtcttaactgcaatacatttaaatgtcactgacttgggtctgggctatgaaaccaaggaggaactgatttttaggtactgcagcggctcttgcgatgcagctgagacaacgtacgacaaaatattgaaaaacttatccagaaatagaaggctggtgagtgacaaagtagggcaggcatgttgcagacccatcgcctttgatgatgacctgtcgtttttagatgataacctggtttaccatattctaagaaagcattccgctaaaaggtgtggatgtatctgactccggctccagagactgctgtgtattgcattcctgctacagtgcaaagaaagggaccaaggttcccaggaaatgtttgcccagaatggaagatgaggaccaaggaggcggaggaggaggaagaagaagaggaggaggaggaggaggaggaggaggaggaggaaggcagccatcatgggagcctggtagagggagatccagctacagacaactggacaggagagagagaaaacagccctctggattctccaggatggcagccgatgtcactagaagctcagggctgatgttcctggttggctattgccaccatttcagctgatacagtccaccatcactgattaccggcgcggttgcggtggatgcacttgaaccaaaccagtgtatctcctgtgatttgttttcatgtgtccgaagacaccagggaaacagagatcctggtgttgttccttgttattacgttttactgctgaaagtaagaggtttatttttctgtcactcagtggagacataccctggaaaggagaggggaaaaaaaaagcaaagatacaagagataatcacctttgcatttgaaagttgaggcccgaggtttactacaaccagcatttttgccaacggttggtgattgatttccatcacggtgtgtggggtgggaagaagttggctaggaaccaaaaaggctgtgctcatgattaaacacaaacctgaaggtatttcctttatgtccttggaaacaggaaacgagttgtggttttcgccagcattcttgtaggagagaatcggggaaggccccgaactgcccccgggcagggagagcccctcaggcctgttggtttacagagagacagatgttacataaccagctccgttgatgcgtggtcaccagtgaccagagaagctactcgatgcaatgcatctgtttcagatacagaaatatagagaagatatttattgaaatttaagttattgttatttattaccgttcactaatgaatttctcttttttcccttatttattaaagtttcttttcaaaggtgccaaagtatatgtgctcgcaaaatgcaaagaaaggtgacaaaaggaaatttgaattgggaacaagggtccatgcttttcaaagtattaaaaagttttttgccaggcaaaaatcacttactttacctttttaagaaaatttgtcattaattttccccagatttcagcatttttcccaatttttatttgtggagcatctcaggcaagccccctttcctggagcagcgtgcagagaccactggcacttgactttatttcttccttgctccattgctgaacagaaatgtcgtgggctccacttcctgttgtctttaagctcttagtcccctccacgtatacctatctgtactatgcataaccatatgtagaaaaggttcagttccttttagtaggtagtcctggatttaatgctgacctaaaagtaatgtcgacaatgctgtcaggtagctgccgttctaccgactccctccatccctgcccacccactgccctcccgagaatatgctggctgcccagtgcagcccgggagacacaggggccttccagaggtagggtctaccaggtcctgtacaacccctgggctgtcaccgggggtcaacagctgctgctcctatatacccaaacacctgacagctccctggggagcagatggctgagaagggtgctgaggaagccatattgggaccagccacagccacacacatggagcctcatacttaggagcgtgctgcctttaaatgaaggtggtcggggccagtgcagcggctcacacccataatcccaacactttggaaagccaaggtgggaggatctcttgaacccaggagtttgagaccagcttgggcaacatagggagaccctgtctctacagaaactttaaaaattaggcaggcatgatggtgcacacctgtggtcccagctactcaagaggctgaaggaggatcacttgagtccagaaggtcgaggctgcagtgagctgtgatcatgccactgcactccagcctaagtgacagtgcggtaccctgtctcaaaaaaaaaaaaaaaaaaaaaaagaggttggagcaggaggaagcataggggcgggaacagccacctcctccatgccctagattgtgaatttatcgggcagccaacacatgtatgacacactaggccctgtattacagcttgttacgcatttcataaaagggattttcattaaggagataatctattactacctaccttagtggctactagtataaaactatgacagatttagcaattaaatgaaatactggcctccatcaaataatcatagtaacaagaagcagcagttaccagacatctgatccccttcccccaaaatacccaaattcttcatggttctgcccttctctgtcctttctgctccccttgctcgcctgggaaatggaggaaaggccttccctctcacactgtcttgggatcttgctgagaattcagactgctcgaaacagtgacaaaccccagccatccagtcattcgtggagcacaatttggatgtggccccaggggcatctgtcccattcagagaaccttggcagtgcgatggccactgttcccaggcttcaacctcagtgaccccccccaacaactccccatggagagtccctgcccaaaaaagctgtaggatccaaggggtgtcaatagctcgttcccggcatcacctacacaccacaagcaggttttaatggaagcaagttgctccaccaaatccacaaaagggtaaagtttgtgatttttctttatcattgcgatcaccatctgataccgtaaggagtgcacttgtttggaagttctgacttctctgatctgtcttggtcgtttgtgttataaaaccaaagttctctacagactttatttttgtacaatatcattttgtaactttttacaaataaaaactcatttctattgc

SEQ ID NO: 5 - DNA, 485 bp, Homo sapiens
  misc_feature 1..485: note = cDNA encoding isoform 1
  source 1..485: mol_type = genomic DNA
caaatatgccagaggattatcctgatcagttcgatgatgtcatggattttattcaagccaccattaaaagactgaaaaggtcaccagataaacaaatggcagtgcttcctagaagagagcggaatcggcaggctgcagctgccaacccagagaattccagaggaaaaggtcggagaggccagaggggcaaaaaccggggttgtgtcttaactgcaatacatttaaatgtcactgacttgggtctgggctatgaaaccaaggaggaactgatttttaggtactgcagcggctcttgcgatgcagctgagacaacgtacgacaaaatattgaaaaacttatccagaaatagaaggctggtgagtgacaaagtagggcaggcatgttgcagacccatcgcctttgatgatgacctgtcgtttttagatgataacctggtttaccatattctaagaaagcattccgctaaaaggtgtggatgtatctga

SEQ ID NO: 6 - protein, 185 aa, Homo sapiens
  REGION 1..185: note = misc_feature - glial cell line-derived neurotrophic factor isoform 2 preproprotein
MKLWDVVAVCLVLLHTASAFPLPAANMPEDYPDQFDDVMDFIQATIKRLKRSPDKQMAVLPRRERNRQAAAANPENSRGKGRRGQRGKNRGCVLTAIHLNVTDLGLGYETKEELIFRYCSGSCDAAETTYDKILKNLSRNRRLVSDKVGQACCRPIAFDDDLSFLDDNLVYHILRKHSAKRCGCI

SEQ ID NO: 7 - protein, 211 aa, Homo sapiens
  REGION 1..211: note = misc_feature - glial cell line-derived neurotrophic factor isoform 1 preproprotein
MKLWDVVAVCLVLLHTASAFPLPAGKRPPEAPAEDRSLGRRRAPFALSSDSNMPEDYPDQFDDVMDFIQATIKRLKRSPDKQMAVLPRRERNRQAAAANPENSRGKGRRGQRGKNRGCVLTAIHLNVTDLGLGYETKEELIFRYCSGSCDAAETTYDKILKNLSRNRRLVSDKVGQACCRPIAFDDDLSFLDDNLVYHILRKHSAKRCGCI

SEQ ID NO: 8 - protein, 228 aa, Homo sapiens
  REGION 1..228: note = misc_feature - glial cell line-derived neurotrophic factor isoform 3 preproprotein
MQSLPNSNGAAAGRDFKMKLWDVVAVCLVLLHTASAFPLPAGKRPPEAPAEDRSLGRRRAPFALSSDSNMPEDYPDQFDDVMDFIQATIKRLKRSPDKQMAVLPRRERNRQAAAANPENSRGKGRRGQRGKNRGCVLTAIHLNVTDLGLGYETKEELIFRYCSGSCDAAETTYDKILKNLSRNRRLVSDKVGQACCRPIAFDDDLSFLDDNLVYHILRKHSAKRCGCI

SEQ ID NO: 9 - protein, 202 aa, Homo sapiens
  REGION 1..202: note = misc_feature - glial cell line-derived neurotrophic factor isoform 4 preproprotein
MQSLPNSNGAAAGRDFKMKLWDVVAVCLVLLHTASAFPLPAANMPEDYPDQFDDVMDFIQATIKRLKRSPDKQMAVLPRRERNRQAAAANPENSRGKGRRGQRGKNRGCVLTAIHLNVTDLGLGYETKEELIFRYCSGSCDAAETTYDKILKNLSRNRRLVSDKVGQACCRPIAFDDDLSFLDDNLVYHILRKHSAKRCGCI

SEQ ID NO: 10 - protein, 211 aa, Mus sp.
  REGION 1..211: note = misc_feature - mouse GDN sequence
MKLWDVVAVCLVLLHTASAFPLPAGKRLLEAPAEDHSLGHRRVPFALTSDSNMPEDYPDQFDDVMDFIQATIKRLKRSPDKQAAALPRRERNRQAAAASPENSRGKGRRGQRGKNRGCVLTAIHLNVTDLGLGYETKEELIFRYCSGSCESAETMYDKILKNLSRSRRLTSDKVGQACCRPVAFDDDLSFLDDNLVYHILRKHSAKRCGCI

SEQ ID NO: 11 - protein, 211 aa, Rattus sp.
  REGION 1..211: note = misc_feature - Rat GDN sequence
MKLWDVVAVCLVLLHTASAFPLPAGKRLLEAPAEDHSLGHRRVPFALTSDSNMPEDYPDQFDDVMDFIQATIKRLKRSPDKQAAALPRRERNRQAAAASPENSRGKGRRGQRGKNRGCVLTAIHLNVTDLGLGYETKEELIFRYCSGSCEAAETMYDKILKNLSRSRRLTSDKVGQACCRPVAFDDDLWFLDDSLVYHILRKHSAKRCGCI

SEQ ID NO: 12 - protein, 196 aa, synthetic construct
  REGION 1..196: note = Consensus sequence derived from human and mouse GDN sequences
MKLWDVVAVCLVLLHTASAFPLPAGKREAPAEDSLGRRPFALSDSNMPEDYPDQFDDVMDFIQATIKRLKRSPDKQALPRRERNRQAAAAPENSRGKGRRGQRGKNRGCVLTAIHLNVTDLGLGYETKEELIFRYCSGSCAETYDKILKNLSRRRLSDKVGQACCRPAFDDDLSFLDDNLVYHILRKHSAKRCGCI

SEQ ID NO: 13 - protein, 195 aa, synthetic construct
  REGION 1..195: note = Consensus sequence derived from human and rat GDN sequences
MKLWDVVAVCLVLLHTASAFPLPAGKREAPAEDSLGRRPFALSDSNMPEDYPDQFDDVMDFIQATIKRLKRSPDKQALPRRERNRQAAAAPENSRGKGRRGQRGKNRGCVLTAIHLNVTDLGLGYETKEELIFRYCSGSCAAETYDKILKNLSRRRLSDKVGQACCRPAFDDDLFLDDLVYHILRKHSAKRCGCI

SEQ ID NO: 14 - DNA, 24 bp, synthetic construct
  misc_feature 1..24: note = Synthetic primer: Fgfr2-Forward
  source 1..24: mol_type = other DNA
ctctctacgtcatagttgaatatg

SEQ ID NO: 15 - DNA, 24 bp, synthetic construct
  misc_feature 1..24: note = Synthetic primer: Fgfr2-Reverse
  source 1..24: mol_type = other DNA
atatccctggccaggccaaagtct

SEQ ID NO: 16 - DNA, 24 bp, synthetic construct
  misc_feature 1..24: note = Synthetic primer: Ret-Forward
  source 1..24: mol_type = other DNA
agatgtttatgaggaagattccta

SEQ ID NO: 17 - DNA, 24 bp, synthetic construct
  misc_feature 1..24: note = Synthetic primer: Ret-Reverse
  source 1..24: mol_type = other DNA
tcctcgctgcagttgtctggcctc

SEQ ID NO: 18 - DNA, 20 bp, synthetic construct
  misc_feature 1..20: note = Synthetic primer: Col4a1-Forward
  source 1..20: mol_type = other DNA
atgccctttctcttctgcaa

SEQ ID NO: 19 - DNA, 24 bp, synthetic construct
  misc_feature 1..24: note = Synthetic primer: Csf1-Forward
  source 1..24: mol_type = other DNA
gatccctgagtctgtcttccacct

SEQ ID NO: 20 - DNA, 25 bp, synthetic construct
  misc_feature 1..25: note = Synthetic primer: Csf1-Reverse
  source 1..25: mol_type = other DNA
cagttccacctgtctgtcctcatcc

SEQ ID NO: 21 - DNA, 24 bp, synthetic construct
  misc_feature 1..24: note = Synthetic primer: Csf1r-Forward
  source 1..24: mol_type = other DNA
gtaaagtggatggccccagagagc

SEQ ID NO: 22 - DNA, 25 bp, synthetic construct
  misc_feature 1..25: note = Synthetic primer: Csf1r-Reverse
  source 1..25: mol_type = other DNA
taggctccaggtcccagcaggactg

SEQ ID NO: 23 - DNA, 26 bp, synthetic construct
  misc_feature 1..26: note = Synthetic primer: c-Myc-Forward
  source 1..26: mol_type = other DNA
cagctcgcccaaatcctgtacctcgt

SEQ ID NO: 24 - DNA, 25 bp, synthetic construct
  misc_feature 1..25: note = Synthetic primer: c-Myc- Reverse
  source 1..25: mol_type = other DNA
cagacaccacatcaatttcttcctc

SEQ ID NO: 25 - DNA, 27 bp, synthetic construct
  misc_feature 1..27: note = Synthetic primer: Notch1 -Forward
  source 1..27: mol_type = other DNA
tgaagaacggagccaacaaggacatgc

SEQ ID NO: 26 - DNA, 27 bp, synthetic construct
  misc_feature 1..27: note = Synthetic primer: Notch1- Reverse
  source 1..27: mol_type = other DNA
gcaatcggtccatgtgatccgtgatgt

SEQ ID NO: 27 - DNA, 20 bp, synthetic construct
  misc_feature 1..20: note = Synthetic primer: Col4a1-Reverse
  source 1..20: mol_type = other DNA
ctgcggaatctgaatggtct

(19) United States
(12) Patent Application Publication
(10) Pub. No.: US 2025/0170214 A1
(43) Pub. Date: May 29, 2025

(54) METHODS FOR PROMOTING WOUND HEALING AND HAIR GROWTH COMPRISING GDNF ADMINISTRATION

(71) Applicant: The Jackson Laboratory, Bar Harbor, ME (US)

(72) Inventors: Robert E. Braun, Bar Harbor, ME (US); Manju Sharma, Bar Harbor, ME (US)

(73) Assignee: The Jackson Laboratory, Bar Harbor, ME (US)

(21) Appl. No.: 19/043,372

(22) Filed: Jan. 31, 2025

Related U.S. Application Data
(63) Continuation of application No. 18/529,590, filed on Dec. 5, 2023.
Application No. 18/529,590 is a continuation of application No. 17/373,877, filed on Jul. 13, 2021, now Pat. No. 11,890,322.
Application No. 17/373,877 is a continuation of application No. 16/452,459, filed on Jun. 25, 2019, now abandoned.

(51) Int. Cl. A61K 38/18; A61K 8/64; A61Q 7/00; C07K 14/475

(52) U.S. Cl. CPC A61K 38/185; A61K 8/64; A61Q 7/00; C07K 14/4756

(57) ABSTRACT

The present invention generally relates to uses of glial cell line-derived growth factor (GDNF) in cutaneous wound healing and hair growth. Methods of effecting hair growth and/or wound healing which feature administration of GDNF, or a biologically active fragment thereof, to subjects, e.g., human subject, are disclosed herein. The invention relates also to formulations and kits for achieving the indicated pharmaceutical advantages.

### RELATED APPLICATIONS

This application claims the benefit of U.S. Provisional Patent Application Ser. No. 61/787,870, filed Mar. 15, 2013. The entire content of the above-referenced patent application is incorporated herein by this reference.

### BACKGROUND OF THE INVENTION

Adult organisms contain several types of cells with remarkable regenerative potential when provided with appropriate chemical or physical stimuli. Wound healing or wound repair is an example of a system where multiple biological pathways are activated during the regeneration of the entire tissue. Skin, the largest organ of the body self-renews throughout adult life. Hair follicles, described as the “bone marrow of the skin”, are a source of numerous growth factors, cytokines and hormones that helps in remodeling the cutaneous environment (Schmidt-Ullrich and Paus (2005), BioEssays 27, 247-261). The role of several growth factors have been reported in the initiation of hair follicle development at embryonic stage but not much is known about their development in adult animals. The role of growth factors in skin biology, in particular, in wound repair or wound healing, has also been reported. However, the exact role of certain growth factors and cytokines in complex processes such as wound repair or wound healing and hair growth remains to be elucidated. Such understanding would greatly facilitate the development of such growth factors and cytokines as pharmaceutical and/or therapeutic agents useful in these complex processes.

### SUMMARY OF THE INVENTION

The present invention is based, at least in part, on the discovery that the cytokine, glial cell line-derived growth factor (GDNF) plays unique roles in the complex processes of wound repair and hair growth. Accordingly, the present invention relates to various pharmaceutical and/or therapeutic methods that feature, in common, administration or delivery of glial cell line-derived growth factor (GDNF) as a biologic active agent. The invention is based, at least in part, on the discovery of several important biological activities of GDNF. In particular, the present inventors have discovered significant regulatory roles for GDNF in biological processes including wound healing and hair growth. Accordingly, in one aspect, the invention relates to methods of promoting wound healing, in particular cutaneous wound healing, wherein said methods feature administration of GDNF, or a biologically active fragment thereof, to a wound site of a subject, e.g., a human subject, in a dose and/or for a time period sufficient to promote wound healing. In particular, the GDNF, or biologically active fragment thereof, is administered in a dose and/or for a time sufficient to promote filling and re-epithelialization of a wound site. In a related aspect, the GDNF, or biologically active fragment thereof, is administered in a dose and/or for a time sufficient to promote reestablishment of a skin barrier at the wound site. In another aspect, the invention relates to methods of promoting hair growth on a subject, wherein said methods feature administration of GDNF, or a biologically active fragment thereof, at site of desired hair growth, in a dose and/or for a time sufficient to promote hair growth on the subject. In another aspect, the invention features pharmaceutical formulations that include a therapeutically effective dose of isolated GDNF, or a biologically active fragment thereof. In yet another aspect, the invention features kits that include said pharmaceutical formulations. In yet another aspect, the invention features the use of a therapeutically effective dose of GDNF, or a biologically active fragment thereof, for promoting wound healing at a wound site in a subject. In yet another aspect, the invention features the use of a pharmaceutically effective dose of GDNF, or a biologically active fragment thereof, for promoting hair growth at a desired site in a subject.

### BRIEF DESCRIPTION OF DRAWINGS

FIG. 1. Mice overexpressing Gdnf under the Cathespin L gene promoter have altered hair follicle development. FVB transgenic mice Tg(Ctsl-Gdnf) have ruffled fur by 3wk of age (A). Skin sections stained with H&E from adult transgenic FVB mouse with more number of hair follicles adjacent to normal hair development (asterisk) compared to skin section from control littermate (B). The transgene overexpressing GDNF in B6C3H background mice also show multiple layers of hair follicles (C). Scale=100 μιη.

FIG. 2. Subcutaneous injection of recombinant GDNF in adult mice results in an increase in hair follicles. Images of skin sections from B6 wild type mice injected with 100 μ† PBS or 10 μg GDNF on alternate days for two weeks. Scale =100 μιη.

FIG. 3. Multiple injections of low dose are more effective than one injection of high concentration of GDNF. H&E image of skin from mouse injected with lOO μl of 0.5 mg/ml (A) or 5 injections of lOO μl of O.1 mg/ml of GDNF on alternate days. Both images are at same magnification. Scale=lOO μm.

FIG. 4. New hair follicle development is specific to GDNF. Adult wild type B6 mice injected with 50 μg FGF9 or GDNF and analyzed after 6 days. There is an increased number of new hair follicles in sections from mice injected with GDNF. Scale=100 um.

FIG. 5. Increased number of hair follicles at a higher dose GDNF. New hair follicles develop as early as day 9 in mice injected with 10 μg of GDNF compared to mice injected with PBS (A). By day 9 there are multiple layers of hair follicles in mice injected with 50 μg GDNF compared ones injected with 10 μg (B). Ki67 labeled cells in hair follicles of skin sections injected with GDNF (C).

FIG. 6. Time course of hair follicle stimulation following GDNF treatment. Hematoxylin and eosin stained section of skin, injected (s.c.) with PBS or 50 μg of GDNF protein and analyzed after 9, 13, and 20 days. There are more numbers of hair follicles at the GDNF injected site compared to PBS. Scale=lOO um

FIG. 7. GDNF seems to be activating Notchl signaling pathway. RT-PCR analysis on RNA extracted after 6 days from skin samples injected with 50 μg of GDNF or PBS in B6 wild type mice. Three fold increase in Notchl but not c-Mye transcript was detected in GDNF injected sample. Transcript of Ret, the tyrosine kinase receptor, protein known to be in GDNF-GFRccl signaling complex, is 4 fold higher in GDNF injected skin.

FIG. 8A-8B. GDNF accelerates wound-healing process in B6 wild type mice. Equal size 3 mm of wounds were made by biopsy punch needle on the dorsal side of adult mice. Wounds were photographed on day 0 and after one week. Only one wound site was injected with GDNF (arrow). H&E stained sections of wound sites injected with PBS or one injection of 0.1 ml of 0.5 mg/ml of GDNF. By one week all layers of skin, epidermis, dermis and hair follicles are present at the wound site injected with GDNF compared to PBS injected sites (A). In the GDNF injected site, a complete layer of epithelium is seen throughout the wound site (arrow) and new blood vessels (arrowheads) after 96 hr were as red blood cells are seen at PBS injected site (asterisk) (B).

FIG. 9. GDNF accelerate the wound healing process in diabetic BKS.Cg-Dock7^{m}+/+Lepr^{d}^{_{b}}/J mice. In the diabetic mice, re-epithelialization (arrow) and neovascularization and blood vessels (arrowhead) formation is observed as early as 96 hr. However, complete wound healing occurs after two weeks in the mice injected with 125 μg of GDNF. Scale=100 um

FIG. 10. GDNF induces blood vessel growth in FVB mice. The figure shows an increased blood vessel growth and branching after injection of 50 μg GDNF (B) in comparison to the PBS control (A).

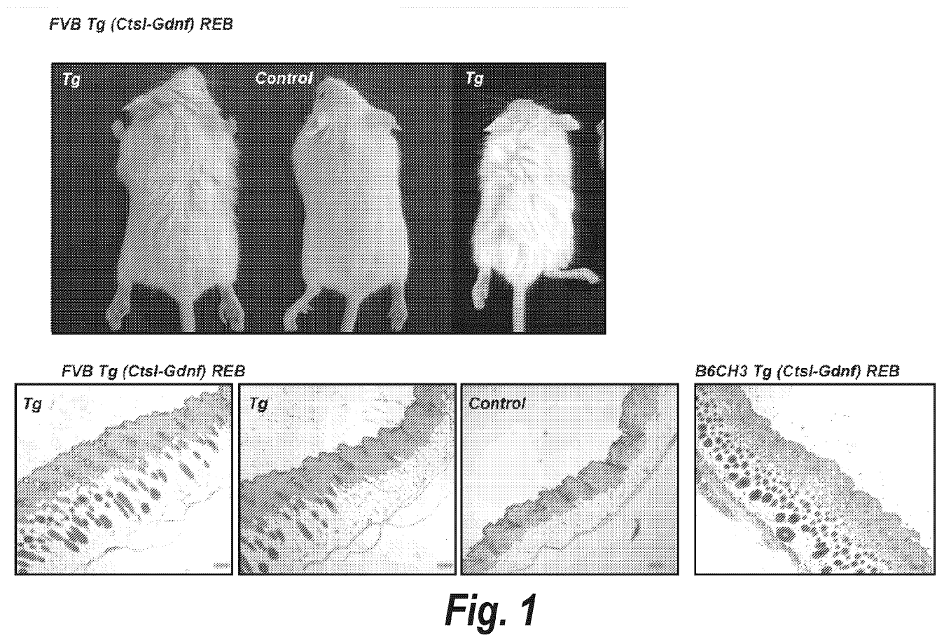

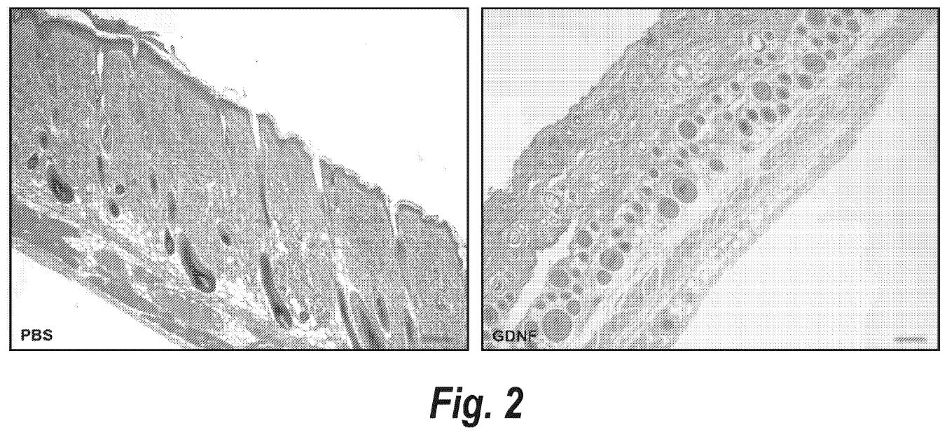

### DETAILED DESCRIPTION OF THE INVENTION

Glial Cell-Derived Neurotrophic Factor (GDNF) is a growth factor with 40% sequence homology to the TGFB superfamily. First identified as survival factor for dopaminergic neurons of mid-brain and shown to be important for the development and maintenance of neural and other tissues (Lin et al 1993, Science 260, 1130-2). GDNF is part of complex that include the high affinity receptor tyrosine kinase, c-RET and glycosyphatidylinositol (GPI-) linked co-receptor, GFRccl that activates specific signaling pathway leading to cell proliferation, survival, and other differentiation effects.

The present invention is based, at least in part, on the discovery of certain key regulatory roles for GDNF in complex processes including wound healing and hair growth. In initial experiments, the effect of GDNF on hair follicle growth was studied. For these studies, purified active recombinant protein expressed in baculovirus (as mammalian proteins expressed in baculovirus are glycosylated) was injected subcutaneously in mice and assayed for hair follicle growth. Preliminary results showed increase in number of hair follicles in mice injected with active form of GDNF compared to mice injected with PBS alone. This finding was very surprising, especially as the effect could be seen after a single injection.

The cutaneous wound healing process involves four steps, first the inflammatory phase followed by proliferative phase, the remodeling step and finally the epithelialization, when new skin is formed. During the proliferative phase extracellular matrix (ECM) components are synthesized and new blood vessels are formed on the matrix. Genes involved in the inflammatory response, angiogenesis and response to wounding were differentially expressed in tissues over-expressing Gdnf. Moreover, when GDNF was injected subcutaneously into mice, smoother skin was discovered. It was therefore predicted that GDNF influences the skin remodeling during wound healing. These findings, in addition to the detailed studies presented in the Examples Section, infra., lead the inventors to propose GDNF as novel factor for hair growth, wound healing, and treatment of scars, wrinkles (anti-aging), in particular, when the GDNF is applied topically to skin.

Prior to describing the invention, it may be helpful to an understanding thereof to set forth definitions of certain terms to be used hereinafter.

### I. Definitions

The terms “homolog,” “paralog,” and “ortholog,” have their art recognized meanings. Typically, a homolog of a given gene product is one of functional similarity as well as sequence similarity. If the homolog is derived from a different organism, the homolog may be referred to as the ortholog. If several homologs exist in a given organism, the homolog may be referred to as a paralog. Typically, the sequence similarity/identity between homologs is at least about 40%, 50%, 60%, 70%, 80%, 90%, or more (or a percentage falling within any interval or range of the foregoing). Methods for determining such similarity/identity are described, infra. Domains {e.g., TGFP-like domains) conserved between homologs can have a sequence similarity/identity of at least about 70%, 80%, 90%, or more. It is understood that when comparing gene product sequence between diverse organisms, for example, flies and humans, sequence similarity between given homologs (e.g., orthologs) across the entire protein sequence may be low. However, if functional complementarity exists, and in addition, if conserved domains exist, e.g., TGFP-like domains, then the gene products being compared can be considered homologs and thus selected as compositions for use in the methods of the invention, as described herein.

The term “bioactive fragment” includes any portion {e.g., a segment of contiguous amino acids) of a polypeptide, e.g., a GDNF polypeptide or ortholog thereof, sufficient to exhibit or exert at least activity of the polypeptide, e.g., at least one GDNF-associated activity including, for example, a growth promoting activity.

The phrase “encodes a gene product” includes the generation of a RNA molecule from a DNA molecule {i.e., a complementary RNA molecule generated from the DNA molecule by the process of transcription) and/or the generation of a polypeptide or protein molecule from an RNA {i.e., by the processes of transcription and translation).

The term “expression” of a gene or nucleic acid encompasses not only cellular gene expression, but also the transcription and translation of nucleic acid(s) in cloning systems and in any other context.

The term “subject”, as used herein, includes living organisms. Examples of subjects include humans, monkeys, cows, sheep, goats, horses, camels, dogs, cats, mice, rats, and transgenic species thereof. Administration of the compositions of the present invention to a subject to be treated can be carried out using known procedures, at dosages and for periods of time effective to modulate hair growth and/or wound healing in the subject as further described herein.

As used herein, the term “isolated” molecule (e.g., isolated protein molecule or isolated peptide) refers to molecules which are substantially free of other cellular material, or culture medium when produced by recombinant techniques, or substantially free of chemical precursors or other chemicals when chemically synthesized.

The term “treatment”, as used herein, is defined as the application or administration of a therapeutic agent to a subject, or application or administration of a therapeutic agent to an isolated tissue or cell line from a subject, who has a disease or disorder, a symptom of a disease or disorder, or a predisposition toward a disease or disorder, with the purpose to cure, heal, alleviate, relieve, alter, remedy, ameliorate, improve or affect the disease or disorder, the symptoms of the disease or disorder, or the predisposition toward a disease or disorder. A therapeutic agent includes, but is not limited to, GDNF peptides, proteins, protein fragments, peptidomimetics, and the like.

The term “effective amount”, as used herein, is defined as that amount necessary or sufficient to treat or prevent a disorder. The “effective amount” can vary depending on such factors as the size and weight of the subject, the type of illness, or the particular agent being administered. One of ordinary skill in the art would be able to study the aforementioned factors and make the determination regarding the effective amount of the agent without undue experimentation.

The term “pharmaceutical composition” as used herein, refers to an agent formulated with one or more compatible solid or liquid filler diluents or encapsulating substances, which are suitable for administration to a human or lower animal.

The term “administering” refers to an administration that is oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation or via an implanted reservoir. The term “parenteral” includes subcutaneous, intravenous, intramuscular, intra-articular, intra-synovial, intrasternal, intrathecal, intrahepatic, intralesional, and intracranial injections or infusion techniques. Typically, the treatment compositions of the present invention are administered topically or by subcutaneous injection.

A “suitable control” or “appropriate control” refers to any control or standard familiar to one of ordinary skill in the art useful for comparison purposes.

The term “cell” refers to any cell of a biological organism. Preferred cells are eukaryotic cells, including but not limited to, animal cells (e.g., mammalian cells, e.g., human or murine cells), nematode cells, plant cells, and yeast. The term includes cell lines, e.g., transformed mammalian cell lines as well as embryonic cells, e.g., embryonic stem cells. Eukaryotic cells responsive to GDNF, or eukaryotic cells involved in hair growth and/or wound repair are preferred cells of the invention. Also contemplated for use in the invention are prokaryotic cells, for use, for example, in methods of manufacturing proteins, e.g., GDNF.

The term “tissue” refers to a collection of cells, usually of different cell types, organized in a manner that imparts complex biological activity.

The term “cell extract” refers to a lysate or acellular preparation of a cell as defined above and can be a crude extract or partially purified as well as comprise additional agents such as recombinant polypeptides, nucleic acids, and/or buffers or stabilizers.

The term “organism” refers to multicellular organisms such as, e.g., C. elegans, Drosophila, mouse, and human.

The terms used herein are not intended to be limiting of the invention.

### II. Glial Cell Line-derived Neurotrophic Factor (GDNF)

Glial cell line-derived neurotrophic factor (or glial cell-derived neurotrophic factor) (GDNF), also known as ATF1, ATF2, HSCR3, and HFB1-GDNF is a distant member of the TGF-β superfamily. Glial-cell-line-derived neurotrophic factor (GDNF) was originally identified as a survival factor for midbrain dopaminergic neurons, and was able to prevent apoptosis of motor neurons induced by axotomy. GDNF and related ligands, neurturin (NRTN), artemin (ARTN) and persephin (PSPN), maintain several neuronal populations in the central nervous systems, including midbrain dopamine neurons and motorneurons. In addition, GDNF, NRTN and ARTN support the survival and regulate the differentiation of many peripheral neurons, including sympathetic, parasympathetic, sensory and enteric neurons. GDNF bas further critical roles outside the nervous system in the regulation of kidney morphogenesis and spermatogenesis.

GDNF family ligands bind to specific GDNF family receptor alpha (GFRalpha) proteins, all of which form receptor complexes and signal through the RET receptor tyrosine kinase (the product of the c-ret (rearranged during transfection) protooncogene). The biological activity of GDNF is mediated by its corresponding high affinity receptor, GDNF family receptor a-1 (GFRa-1) which functions as part of a receptor complex with the intracellular-signaling component, RET. The mature form of the protein is considered a ligand for RET. GDNF also shows lower-affinity interactions with GFRa-2. The biology of GDNF signaling is much more complex than originally assumed. The neurotrophic effect of GDNF, except in motorneurons, requires the presence of TGF-β, which activates the transport of GFRa1 to the cell membrane. GDNF can also signal RET independently through GFRla. Upon ligand binding. GDNF in complex with GFRal may interact with heparan sulphate glycosaminoglycans to activate the Met receptor tyrosine kinase through cytoplasmic Src-family kinases. GDNF family ligands also signal through the neural cell adhesion molecule NCAM. In cells lacking RET, GDNF binds with high affinity to the NCAM and GFRal complex, which activates Fyn and FAK.

This GDNF gene encodes a highly conserved neurotrophic factor. The encoded protein is processed to a mature secreted form that exists as a homodimer. Multiple transcript variants encoding different isoforms have been found for the human GDNF gene. Transcript variant (1) differs in the 5′ UTR, representing use of an alternate promoter, and a downstream start codon, compared to variant 3. The resulting isoform (1) has a shorter N-terminus, compared to isoform 3. Transcript variant (2) also differs in the 5′ UTR, and represents use of an alternate promoter, uses a downstream start codon, and uses an alternate in-frame splice site in the coding region, compared to variant 3. The resulting isoform (2) has a shorter N-terminus and lacks an internal segment, compared to isoform 3. Transcript variant (3) represents the longest transcript and encodes the longest isoform (3). Transcript Variant: This variant (4) uses an alternate in-frame splice site in the coding region, compared to variant 3. The resulting isoform (4) lacks an internal segment, compared to isoform 3.

The nucleic acids encoding the human GDNF isoforms are as follows:

>gi |299473777 |ref |NM_000514.3| Homo sapiens glial cell derived
neurotrophic factor (GDNF) , transcript variant 1, mRNA
(SEQ ID NO: 1)
CCGCCTCCAGCGCGCCCTTGCTGCCCCGCGCGACCCCAGGATTGCGAACTCTTGCCCCTGACCTGTTGGG
CGGGGCTCCGCGCTCCAGCCATCAGCCCGGATGGGTCTCCTGGCTGGGACTTGGGGCACCTGGAGTTAAT
GTCCAACCTAGGGTCTGCGGAGACCCGATCCGAGGTGCCGCCGCCGGACGGGACTTTAAGATGAAGTTAT
GGGATGTCGTGGCTGTCTGCCTGGTGCTGCTCCACACCGCGTCCGCCTTCCCGCTGCCCGCCGGTAAGAG
GCCTCCCGAGGCGCCCGCCGAAGACCGCTCCCTCGGCCGCCGCCGCGCGCCCTTCGCGCTGAGCAGTGAC
TCAAATATGCCAGAGGATTATCCTGATCAGTTCGATGATGTCATGGATTTTATTCAAGCCACCATTAAAA
GACTGAAAAGGTCACCAGATAAACAAATGGCAGTGCTTCCTAGAAGAGAGCGGAATCGGCAGGCTGCAGC
TGCCAACCCAGAGAATTCCAGAGGAAAAGGTCGGAGAGGCCAGAGGGGCAAAAACCGGGGTTGTGTCTTA
ACTGCAATACATTTAAATGTCACTGACTTGGGTCTGGGCTATGAAACCAAGGAGGAACTGATTTTTAGGT
ACTGCAGCGGCTCTTGCGATGCAGCTGAGACAACGTACGACAAAATATTGAAAAACTTATCCAGAAATAG
AAGGCTGGTGAGTGACAAAGTAGGGCAGGCATGTTGCAGACCCATCGCCTTTGATGATGACCTGTCGTTT
TTAGATGATAACCTGGTTTACCATATTCTAAGAAAGCATTCCGCTAAAAGGTGTGGATGTATCTGACTCC
GGCTCCAGAGACTGCTGTGTATTGCATTCCTGCTACAGTGCAAAGAAAGGGACCAAGGTTCCCAGGAAAT
GTTTGCCCAGAA TGGAAGA TGAGGA CCAAG GAGGCGGAGGAGGAG GAAGAAGAAGAGGAGGAGGAGGAGG
AGGAGGAGGAGGAGGAGGAAGGCAGCCATCATGGGAGCCTGGTAGAGGGAGATCCAGCTACAGACAACTG
GACAGGAGAGAGAGAAAACAGCCCTCTGGATTCTCCAGGATGGCAGCCGATGTCACTAGAAGCTCAGGGC
TGATGTTCCTGGTTGGCTATTGCCACCATTTCAGCTGATACAGTCCACCATCACTGATTACCGGCGCGGT
TGCGGTGGATGCACTTGAACCAAACCAGTGTATCTCCTGTGATTTGTTTTCATGTGTCCGAAGACACCAG
GGAAACAGAGATCCTGGTGTTGTTCCTTGTTATTACGTTTTACTGCTGAAAGTAAGAGGTTTATTTTTCT
GTCACTCAGTGGAGACATACCCTGGAAAGGAGAGGGGAAAAAAAAAGCAAAGATACAAGAGATAATCACC
TTTGCATTTGAAAGTTGAGGCCCGAGGTTTACTACAACCAGCATTTTTGCCAACGGTTGGTGATTGATTT
CCATCACGGTGTGTGGGGTGGGAAGAAGTTGGCTAGGAACCAAAAAGGCTGTGCTCATGATTAAACACAA
ACCTGAAGGTATTTCCTTTATGTCCTTGGAAACAGGAAACGAGTTGTGGTTTTCGCCAGCATTCTTGTAG
GAGAGAATCGGGGAAGGCCCCGAACTGCCCCCGGGCAGGGAGAGCCCCTCAGGCCTGTTGGTTTACAGAG
AGACAGATGTTACATAACCAGCTCCGTTGATGCGTGGTCACCAGTGACCAGAGAAGCTACTCGATGCAAT
GCATCTGTTTCAGA TACAGAAAT A TAGAGAAGA TA TTTA TTGAAAT TTAAG TTATTGTTATTTA TTACCG
TTCACTAATGAATTTCTCTTTTTTCCCTTATTTATTAAAGTTTCTTTTCAAAGGTGCCAAAGTATATGTG
CTCGCAAAATGCAAAGAAAGGTGACAAAAGGAAATTTGAATTGGGAACAAGGGTCCATGCTTTTCAAAGT
ATTAAAAAGTTTTTTGCCAGGCAAAAATCACTTACTTTACCTTTTTAAGAAAATTTGTCATTAATTTTCC
CCAGATTTCAGCATTTTTCCCAATTTTTATTTGTGGAGCATCTCAGGCAAGCCCCCTTTCCTGGAGCAGC
GTGCAGAGACCACTGGCACTTGACTTTATTTCTTCCTTGCTCCATTGCTGAACAGAAATGTCGTGGGCTC
CACTTCCTGTTGTCTTTAAGCTCTTAGTCCCCTCCACGTATACCTATCTGTACTATGCATAACCATATGT
AGAAAAGGTTCAGTTCCTTTTAGTAGGTAGTCCTGGATTTAATGCTGACCTAAAAGTAATGTCGACAATG
CTGTCAGGTAGCTGCCGTTCTACCGACTCCCTCCATCCCTGCCCACCCACTGCCCTCCCGAGAATATGCT
GGCTGCCCAGTGCAGCCCGGGAGACACAGGGGCCTTCCAGAGGTAGGGTCTACCAGGTCCTGTACAACCC
CTGGGCTGTCACCGGGGGTCAACAGCTGCTGCTCCTATATACCCAAACACCTGACAGCTCCCTGGGGAGC
AGATGGCTGAGAAGGGTGCTGAGGAAGCCATATTGGGACCAGCCACAGCCACACACATGGAGCCTCATAC
TTAGGAGCGTGCTGCCTTTAAATGAAGGTGGTCGGGGCCAGTGCAGCGGCTCACACCCATAATCCCAACA
CTTTGGAAAGCCAAGGTGGGAGGATCTCTTGAACCCAGGAGTTTGAGACCAGCTTGGGCAACATAGGGAG
ACCCTGTCTCTACAGAAACTTTAAAAATTAGGCAGGCATGATGGTGCACACCTGTGGTCCCAGCTACTCA
AGAGGCTGAAGGAGGATCACTTGAGTCCAGAAGGTCGAGGCTGCAGTGAGCTGTGATCATGCCACTGCAC
TCCAGCCTAAGTGACAGTGCGGTACCCTGTCTCAAAAAAAAAAAAAAAAAAAAAAAGAGGTTGGAGCAGG
AGGAAGCATAGGGGCGGGAACAGCCACCTCCTCCATGCCCTAGATTGTGAATTTATCGGGCAGCCAACAC
ATGTATGACACACTAGGCCCTGTATTACAGCTTGTTACGCATTTCATAAAAGGGATTTTCATTAAGGAGA
TAATCTATTACTACCTACCTTAGTGGCTACTAGTATAAAACTATGACAGATTTAGCAATTAAATGAAATA
CTGGCCTCCATCAAATAATCATAGTAACAAGAAGCAGCAGTTACCAGACATCTGATCCCCTTCCCCCAAA
ATACCCAAATTCTTCATGGTTCTGCCCTTCTCTGTCCTTTCTGCTCCCCTTGCTCGCCTGGGAAATGGAG
GAAAGGCCTTCCCTCTCACACTGTCTTGGGATCTTGCTGAGAATTCAGACTGCTCGAAACAGTGACAAAC
CCCAGCCATCCAGTCATTCGTGGAGCACAATTTGGATGTGGCCCCAGGGGCATCTGTCCCATTCAGAGAA
CCTTGGCAGTGCGATGGCCACTGTTCCCAGGCTTCAACCTCAGTGACCCCCCCCAACAACTCCCCATGGA
GAGTCCCTGCCCAAAAAAGCTGTAGGATCCAAGGGGTGTCAATAGCTCGTTCCCGGCATCACCTACACAC
CACAAGCAGGTTTTAATGGAAGCAAGTTGCTCCACCAAATCCACAAAAGGGTAAAGTTTGTGATTTTTCT
TTATCATTGCGATCACCATCTGATACCGTAAGGAGTGCACTTGTTTGGAAGTTCTGACTTCTCTGATCTG
TCTTGGTCGTTTGTGTTATAAAACCAAAGTTCTCTACAGACTTTATTTTTGTACAATATCATTTTGTAAC
TTTTTA CAAATAAAAAC TCATTTCTATTGC
>gi |299473776 |ref |NM_199231.2| Homo sapiens glial cell derived
neurotrophic factor (GDNF) , transcript variant 2, mRNA
(SEQ ID NO: 2)
CATACAGGCCAAAAGTCTCCAAGTCCCTGCTAACTTCTTGCTCTCGCAACAGAATACCTATTTAGGTGGG
AAGAATGAGGTGTGGGCGGCAGGCTGGGTGCCGCCGCCGGACGGGACTTTAAGATGAAGTTATGGGATGT
CGTGGCTGTCTGCCTGGTGCTGCTCCACACCGCGTCCGCCTTCCCGCTGCCCGCCGCAAATATGCCAGAG
GATTATCCTGATCAGTTCGA TGA TGTCA TGGATTTTATTCAAGC CACCATTAAAAG ACTGAAAAGG TCAC
CAGATAAACAAATGGCAGTGCTTCCTAGAAGAGAGCGGAATCGGCAGGCTGCAGCTGCCAACCCAGAGAA
TTCCAGAGGAAAAGGTCGGAGAGGCCAGAGGGGCAAAAACCGGGGTTGTGTCTTAACTGCAATACATTTA
AATGTCACTGACTTGGGTCTGGGCTATGAAACCAAGGAGGAACTGATTTTTAGGTACTGCAGCGGCTCTT
GCGATGCAGCTGAGACAACGTACGACAAAATATTGAAAAACTTATCCAGAAATAGAAGGCTGGTGAGTGA
CAAAGTAGGGCAGGCATGTTGCAGACCCATCGCCTTTGATGATGACCTGTCGTTTTTAGATGATAACCTG
GTTTACCATATTCTAAGAAAGCATTCCGCTAAAAGGTGTGGATGTATCTGACTCCGGCTCCAGAGACTGC
TGTGTATTGCATTCCTGCTACAGTGCAAAGAAAGGGACCAAGGTTCCCAGGAAATGTTTGCCCAGAATGG
AAGA TGAGGA CCAAG GAGGCGGAGGAGGAG GAAGAAGAAGAGGAGGAGGAGGAGGAGGAGGAGGAGGAGG
AGGAAGGCAGCCATCATGGGAGCCTGGTAGAGGGAGATCCAGCTACAGACAACTGGACAGGAGAGAGAGA
AAACAGCCCTCTGGATTCTCCAGGATGGCAGCCGATGTCACTAGAAGCTCAGGGCTGATGTTCCTGGTTG
GCTATTGCCACCATTTCAGCTGATACAGTCCACCATCACTGATTACCGGCGCGGTTGCGGTGGATGCACT
TGAACCAAACCAGTGTATCTCCTGTGATTTGTTTTCATGTGTCCGAAGACACCAGGGAAACAGAGATCCT
GGTGTTGTTCCTTGTTATTACGTTTTACTGCTGAAAGTAAGAGGTTTATTTTTCTGTCACTCAGTGGAGA
CATACCCTGGAAAGGAGAGGGGAAAAAAAAAGCAAAGATACAAGAGATAATCACCTTTGCATTTGAAAGT
TGAGGCCCGAGGTTTACTACAACCAGCATTTTTGCCAACGGTTGGTGATTGATTTCCATCACGGTGTGTG
GGGTGGGAAGAAGTTGGCTAGGAACCAAAAAGGCTGTGCTCATGATTAAACACAAACCTGAAGGTATTTC
CTTTATGTCCTTGGAAACAGGAAACGAGTTGTGGTTTTCGCCAGCATTCTTGTAGGAGAGAATCGGGGAA
GGCCCCGAACTGCCCCCGGGCAGGGAGAGCCCCTCAGGCCTGTTGGTTTACAGAGAGACAGATGTTACAT
AACCAGCTCCGTTGATGCGTGGTCACCAGTGACCAGAGAAGCTACTCGATGCAATGCATCTGTTTCAGAT
ACAGAAATATAGAGAAGATATTTATTGAAAT TTAAGTTATTGTTATTTA TTACCGTTCACTAATGAAT TT
CTCTTTTTTCCCTTATTTATTAAAGTTTCTTTTCAAAGGTGCCAAAGTATATGTGCTCGCAAAATGCAAA
GAAAGGTGACAAAAGGAAATTTGAATTGGGAACAAGGGTCCATGCTTTTCAAAGTATTAAAAAGTTTTTT
GCCAGGCAAAAATCACTTACTTTACCTTTTTAAGAAAATTTGTCATTAATTTTCCCCAGATTTCAGCATT
TTTCCCAATTTTTATTTGTGGAGCATCTCAGGCAAGCCCCCTTTCCTGGAGCAGCGTGCAGAGACCACTG
GCACTTGACTTTATTTCTTCCTTGCTCCATTGCTGAACAGAAATGTCGTGGGCTCCACTTCCTGTTGTCT
TTAAGC TCTTAGTCCCCTCCACGTATACCTATCTGTACTATGCATAACCATATGTAGAAAAGG TTCAGTT
CCTTTTAGTAGGTAGTCCTGGATTTAATGCTGACCTAAAAGTAATGTCGACAATGCTGTCAGGTAGCTGC
CGTTCTACCGACTCCCTCCATCCCTGCCCACCCACTGCCCTCCCGAGAATATGCTGGCTGCCCAGTGCAG
CCCGGGAGACACAGGGGCCTTCCAGAGGTAGGGTCTACCAGGTCCTGTACAACCCCTGGGCTGTCACCGG
GGGTCAACAGCTGCTGCTCCTATATACCCAAACACCTGACAGCTCCCTGGGGAGCAGATGGCTGAGAAGG
GTGCTGAGGAAGCCATATTGGGACCAGCCACAGCCACACACATGGAGCCTCATACTTAGGAGCGTGCTGC
CTTTAAATGAAGGTGGTCGGGGCCAGTGCAGCGGCTCACACCCATAATCCCAACACTTTGGAAAGCCAAG
GTGGGAGGATCTCTTGAACCCAGGAGTTTGAGACCAGCTTGGGCAACATAGGGAGACCCTGTCTCTACAG
AAACTTTAAAAATTAGGCAGGCATGATGGTGCACACCTGTGGTCCCAGCTACTCAAGAGGCTGAAGGAGG
ATCACTTGAGTCCAGAAGGTCGAGGCTGCAGTGAGCTGTGATCATGCCACTGCACTCCAGCCTAAGTGAC
AGTGCGGTACCCTGTCTCAAAAAAAAAAAAAAAAAAAAAAAGAGGTTGGAGCAGGAGGAAGCATAGGGGC
GGGAACAGCCACCTCCTCCATGCCCTAGATTGTGAATTTATCGGGCAGCCAACACATGTATGACACACTA
GGCCCTGTATTACAGCTTGTTACGCATTTCATAAAAGGGATTTTCATTAAGGAGATAATCTATTACTACC
TACCTTAGTGGCTACTAGTATAAAACTATGACAGATTTAGCAATTAAATGAAATACTGGCCTCCATCAAA
TAATCATAGTAACAAGAAGCAGCAGTTACCAGACATCTGATCCCCTTCCCCCAAAATACCCAAATTCTTC
ATGGTTCTGCCCTTCTCTGTCCTTTCTGCTCCCCTTGCTCGCCTGGGAAATGGAGGAAAGGCCTTCCCTC
TCACACTGTCTTGGGATCTTGCTGAGAATTCAGACTGCTCGAAACAGTGACAAACCCCAGCCATCCAGTC
ATTCGTGGAGCACAATTTGGATGTGGCCCCAGGGGCATCTGTCCCATTCAGAGAACCTTGGCAGTGCGAT
GGCCACTGTTCCCAGGCTTCAACCTCAGTGACCCCCCCCAACAACTCCCCATGGAGAGTCCCTGCCCAAA
AAAGCTGTAGGATCCAAGGGGTGTCAATAGCTCGTTCCCGGCATCACCTACACACCACAAGCAGGTTTTA
ATGGAAGCAAGTTGCTCCACCAAATCCACAAAAGGGTAAAGTTTGTGATTTTTCTTTATCATTGCGATCA
CCATCTGATACCGTAAGGAGTGCACTTGTTTGGAAGTTCTGACTTCTCTGATCTGTCTTGGTCGTTTGTG
TTATAAAACCAAAGTTCTCTACAGACTTTATTTTTGTACAATATCATTTTGTAACTTTTTACAAATAAAA
ACTCATTTCTATTGC
>gi |299473778 |ref |NM_001190468.1| Homo sapiens glial cell derived
neurotrophic factor (GDNF), transcript variant 3, mRNA
(SEQ ID NO: 3)
CCAAAGCGTCCGAGACTGGGTACAGTCGTCCAGGCGTGACGGGGGCGCGGGGAGCCAGTGACTCCTCTGG
GAGGGGAAGGGATTAGGGCCAGAATCTCTCAAAGGTGCAAAAATCCAGTCAAGAGAGGGTTTTCGGGTAT
ACCACGGAGGATTAAAACTTTCAAGACAAATGCAGTCTTTGCCTAACAGCAATGGTGCCGCCGCCGGACG
GGACTTTAAGATGAAGTTATGGGATGTCGTGGCTGTCTGCCTGGTGCTGCTCCACACCGCGTCCGCCTTC
CCGCTGCCCGCCGGTAAGAGGCCTCCCGAGGCGCCCGCCGAAGACCGCTCCCTCGGCCGCCGCCGCGCGC
CCTTCGCGCTGAGCAGTGACTCAAATATGCCAGAGGATTATCCTGATCAGTTCGATGATGTCATGGATTT
TATTCAAGCCACCATTAAAAGACTGAAAAGGTCACCAGATAAACAAATGGCAGTGCTTCCTAGAAGAGAG
CGGAATCGGCAGGCTGCAGCTGCCAACCCAGAGAATTCCAGAGGAAAAGGTCGGAGAGGCCAGAGGGGCA
AAAACCGGGGTTGTGTCTTAACTGCAATACATTTAAATGTCACTGACTTGGGTCTGGGCTATGAAACCAA
GGAGGAACTGATTTTTAGGTACTGCAGCGGCTCTTGCGATGCAGCTGAGACAACGTACGACAAAATATTG
AAAAACTTATCCAGAAATAGAAGGCTGGTGAGTGACAAAGTAGGGCAGGCATGTTGCAGACCCATCGCCT
TTGA TGA TGA CCTGTCGTTTTTAGA TGATAACCTGGTTTACCATATTCTAAGAAAGCA TTCCGCTAAAAG
GTGTGGATGTATCTGACTCCGGCTCCAGAGACTGCTGTGTATTGCATTCCTGCTACAGTGCAAAGAAAGG
GACCAAGGTTCCCAGGAAATGTTTGCCCAGAATGGAAGATGAGGACCAAGGAGGCGGAGGAGGAGGAAGA
AGAAGAGGAGGAGGAGGAGGAGGAGGAGGAGGAGGAGGAAGGCAGCCATCATGGGAGCCTGGTAGAGGGA
GATCCAGCTACAGACAACTGGACAGGAGAGAGAGAAAACAGCCCTCTGGATTCTCCAGGATGGCAGCCGA
TGTCACTAGAAGCTCAGGGCTGATGTTCCTGGTTGGCTATTGCCACCATTTCAGCTGATACAGTCCACCA
TCACTGATTACCGGCGCGGTTGCGGTGGATGCACTTGAACCAAACCAGTGTATCTCCTGTGATTTGTTTT
CATGTGTCCGAAGACACCAGGGAAACAGAGATCCTGGTGTTGTTCCTTGTTATTACGTTTTACTGCTGAA
AGTAAGAGGTTTATTTTTCTGTCACTCAGTGGAGACATACCCTGGAAAGGAGAGGGGAAAAAAAAAGCAA
AGATACAAGAGATAATCACCTTTGCATTTGAAAGTTGAGGCCCGAGGTTTACTACAACCAGCATTTTTGC
CAACGGTTGGTGATTGATTTCCATCACGGTGTGTGGGGTGGGAAGAAGTTGGCTAGGAACCAAAAAGGCT
GTGCTCATGATTAAACACAAACCTGAAGGTATTTCCTTTATGTCCTTGGAAACAGGAAACGAGTTGTGGT
TTTCGCCAGCATTCTTGTAGGAGAGAATCGGGGAAGGCCCCGAACTGCCCCCGGGCAGGGAGAGCCCCTC
AGGCCTGTTGGTTTACAGAGAGACAGATGTTACATAACCAGCTCCGTTGATGCGTGGTCACCAGTGACCA
GAGAAGC TACTCGA TGCAATGCATCTGTTTCAGATACAGAAATATAGAGAAGA TA TTTA TTGAAAT TTAA
GTTATTGTTATTTATTACCGTTCACTAATGAATTTCTCTTTTTTCCCTTATTTATTAAAGTTTCTTTTCA
AAGGTGCCAAAGTATATGTGCTCGCAAAATGCAAAGAAAGGTGACAAAAGGAAATTTGAATTGGGAACAA
GGGTCCATGCTTTTCAAAGTATTAAAAAGTTTTTTGCCAGGCAAAAATCACTTACTTTACCTTTTTAAGA
AAATTTGTCATTAATTTTCCCCAGATTTCAGCATTTTTCCCAATTTTTATTTGTGGAGCATCTCAGGCAA
GCCCCCTTTCCTGGAGCAGCGTGCAGAGACCACTGGCACTTGACTTTATTTCTTCCTTGCTCCATTGCTG
AACAGAAATGTCGTGGGCTCCACTTCCTGTTGTCTTTAAGCTCTTAGTCCCCTCCACGTATACCTATCTG
TACTATGCATAACCATATGTAGAAAAGGTTCAGTTCCTTTTAGTAGGTAGTCCTGGATTTAATGCTGACC
TAAAAGTAATGTCGACAATGCTGTCAGGTAGCTGCCGTTCTACCGACTCCCTCCATCCCTGCCCACCCAC
TGCCCTCCCGAGAATATGCTGGCTGCCCAGTGCAGCCCGGGAGACACAGGGGCCTTCCAGAGGTAGGGTC
TACCAGGTCCTGTACAACCCCTGGGCTGTCACCGGGGGTCAACAGCTGCTGCTCCTATATACCCAAACAC
CTGACAGCTCCCTGGGGAGCAGATGGCTGAGAAGGGTGCTGAGGAAGCCATATTGGGACCAGCCACAGCC
ACACACATGGAGCCTCATACTTAGGAGCGTGCTGCCTTTAAATGAAGGTGGTCGGGGCCAGTGCAGCGGC
TCACACCCATAATCCCAACACTTTGGAAAGCCAAGGTGGGAGGATCTCTTGAACCCAGGAGTTTGAGACC
AGCTTGGGCAACATAGGGAGACCCTGTCTCTACAGAAACTTTAAAAATTAGGCAGGCATGATGGTGCACA
CCTGTGGTCCCAGCTACTCAAGAGGCTGAAGGAGGATCACTTGAGTCCAGAAGGTCGAGGCTGCAGTGAG
CTGTGATCATGCCACTGCACTCCAGCCTAAGTGACAGTGCGGTACCCTGTCTCAAAAAAAAAAAAAAAAA
AAAAAAGAGGTTGGAGCAGGAGGAAGCATAGGGGCGGGAACAGCCACCTCCTCCATGCCCTAGATTGTGA
ATTTATCGGGCAGCCAACACATGTATGACACACTAGGCCCTGTATTACAGCTTGTTACGCATTTCATAAA
AGGGATTTTCATTAAGGAGATAATCTATTACTACCTACCTTAGTGGCTACTAGTATAAAACTATGACAGA
TTTAGCAATTAAATGAAATACTGGCCTCCATCAAATAATCATAGTAACAAGAAGCAGCAGTTACCAGACA
TCTGATCCCCTTCCCCCAAAATACCCAAATTCTTCATGGTTCTGCCCTTCTCTGTCCTTTCTGCTCCCCT
TGCTCGCCTGGGAAATGGAGGAAAGGCCTTCCCTCTCACACTGTCTTGGGATCTTGCTGAGAATTCAGAC
TGCTCGAAACAGTGACAAACCCCAGCCATCCAGTCATTCGTGGAGCACAATTTGGATGTGGCCCCAGGGG
CATCTGTCCCATTCAGAGAACCTTGGCAGTGCGATGGCCACTGTTCCCAGGCTTCAACCTCAGTGACCCC
CCCCAACAACTCCCCATGGAGAGTCCCTGCCCAAAAAAGCTGTAGGATCCAAGGGGTGTCAATAGCTCGT
TCCCGGCATCACCTACACACCACAAGCAGGTTTTAATGGAAGCAAGTTGCTCCACCAAATCCACAAAAGG
GTAAAGTTTGTGATTTTTCTTTATCATTGCGATCACCATCTGATACCGTAAGGAGTGCACTTGTTTGGAA
GTTCTGACTTCTCTGATCTGTCTTGGTCGTTTGTGTTATAAAACCAAAGTTCTCTACAGACTTTATTTTT
GTACAATATCATTTTGTAACTTTTTACAAATAAAAACTCATTTCTATTGC
>gi |299473780 |ref |NM_001190469.1| Homo sapiens glial cell derived
neurotrophic factor (GDNF), transcript variant 4, mRNA
(SEQ ID NO: 4)
CCAAAGCGTCCGAGACTGGGTACAGTCGTCCAGGCGTGACGGGGGCGCGGGGAGCCAGTGACTCCTCTGG
GAGGGGAAGGGATTAGGGCCAGAATCTCTCAAAGGTGCAAAAATCCAGTCAAGAGAGGGTTTTCGGGTAT
ACCACGGAGGATTAAAACTTTCAAGACAAATGCAGTCTTTGCCTAACAGCAATGGTGCCGCCGCCGGACG
GGACTTTAAGATGAAGTTATGGGATGTCGTGGCTGTCTGCCTGGTGCTGCTCCACACCGCGTCCGCCTTC
CCGCTGCCCGCCGCAAATATGCCAGAGGATTATCCTGATCAGTTCGATGATGTCATGGATTTTATTCAAG
CCACCATTAAAAGACTGAAAAGGTCACCAGATAAACAAATGGCAGTGCTTCCTAGAAGAGAGCGGAATCG
GCAGGCTGCAGCTGCCAACCCAGAGAATTCCAGAGGAAAAGGTCGGAGAGGCCAGAGGGGCAAAAACCGG
GGTTGTGTCTTAACTGCAATACATTTAAATGTCACTGACTTGGGTCTGGGCTATGAAACCAAGGAGGAAC
TGATTTTTAGGTACTGCAGCGGCTCTTGCGATGCAGCTGAGACAACGTACGACAAAATATTGAAAAACTT
ATCCAGAAATAGAAGGCTGGTGAGTGACAAAGTAGGGCAGGCATGTTGCAGACCCATCGCCTTTGATGAT
GACCTGTCGTTTTTAGATGATAACCTGGTTTACCATATTCTAAGAAAGCATTCCGCTAAAAGGTGTGGAT
GTATCTGACTCCGGCTCCAGAGACTGCTGTGTATTGCATTCCTGCTACAGTGCAAAGAAAGGGACCAAGG
TTCCCAGGAAATGTTTGCCCAGAATGGAAGATGAGGACCAAGGAGGCGGAGGAGGAGGAAGAAGAAGAGG
AGGAGGAGGAGGAGGAGGAGGAGGAGGAGGAAGGCAGCCATCATGGGAGCCTGGTAGAGGGAGATCCAGC
TACAGACAACTGGACAGGAGAGAGAGAAAACAGCCCTCTGGATTCTCCAGGATGGCAGCCGATGTCACTA
GAAGCTCAGGGCTGATGTTCCTGGTTGGCTATTGCCACCATTTCAGCTGATACAGTCCACCATCACTGAT
TACCGGCGCGGTTGCGGTGGATGCACTTGAACCAAACCAGTGTATCTCCTGTGATTTGTTTTCATGTGTC
CGAAGACACCAGGGAAACAGAGATCCTGGTGTTGTTCCTTGTTATTACGTTTTACTGCTGAAAGTAAGAG
GTTTATTTTTCTGTCACTCAGTGGAGACATACCCTGGAAAGGAGAGGGGAAAAAAAAAGCAAAGATACAA
GAGATAATCACCTTTGCATTTGAAAGTTGAGGCCCGAGGTTTACTACAACCAGCATTTTTGCCAACGGTT
GGTGATTGATTTCCATCACGGTGTGTGGGGTGGGAAGAAGTTGGCTAGGAACCAAAAAGGCTGTGCTCAT
GATTAAACACAAACCTGAAGGTATTTCCTTTATGTCCTTGGAAACAGGAAACGAGTTGTGGTTTTCGCCA
GCATTCTTGTAGGAGAGAATCGGGGAAGGCCCCGAACTGCCCCCGGGCAGGGAGAGCCCCTCAGGCCTGT
TGGTTTACAGAGAGACAGATGTTACATAACCAGCTCCGTTGATGCGTGGTCACCAGTGACCAGAGAAGCT
ACTCGA TGCAATGCATCTGTTTCAGATACAGAAATATAGAGAAGATATTTA TTGAAAT TTAAG TTATTGT
TATTTATTACCGTTCACTAATGAATTTCTCTTTTTTCCCTTATTTATTAAAGTTTCTTTTCAAAGGTGCC
AAAGTATATGTGCTCGCAAAATGCAAAGAAAGGTGACAAAAGGAAATTTGAATTGGGAACAAGGGTCCAT
GCTTTTCAAAGTATTAAAAAGTTTTTTGCCAGGCAAAAATCACTTACTTTACCTTTTTAAGAAAATTTGT
CATTAATTTTCCCCAGATTTCAGCATTTTTCCCAATTTTTATTTGTGGAGCATCTCAGGCAAGCCCCCTT
TCCTGGAGCAGCGTGCAGAGACCACTGGCACTTGACTTTATTTCTTCCTTGCTCCATTGCTGAACAGAAA
TGTCGTGGGCTCCACTTCCTGTTGTCTTTAAGCTCTTAGTCCCCTCCACGTATACCTATCTGTACTATGC
ATAACCATATGTAGAAAAGGTTCAGTTCCTTTTAGTAGGTAGTCCTGGATTTAATGCTGACCTAAAAGTA
ATGTCGACAATGCTGTCAGGTAGCTGCCGTTCTACCGACTCCCTCCATCCCTGCCCACCCACTGCCCTCC
CGAGAATATGCTGGCTGCCCAGTGCAGCCCGGGAGACACAGGGGCCTTCCAGAGGTAGGGTCTACCAGGT
CCTGTACAACCCCTGGGCTGTCACCGGGGGTCAACAGCTGCTGCTCCTATATACCCAAACACCTGACAGC
TCCCTGGGGAGCAGATGGCTGAGAAGGGTGCTGAGGAAGCCATATTGGGACCAGCCACAGCCACACACAT
GGAGCCTCATACTTAGGAGCGTGCTGCCTTTAAATGAAGGTGGTCGGGGCCAGTGCAGCGGCTCACACCC
ATAATCCCAACACTTTGGAAAGCCAAGGTGGGAGGATCTCTTGAACCCAGGAGTTTGAGACCAGCTTGGG
CAACATAGGGAGACCCTGTCTCTACAGAAACTTTAAAAATTAGGCAGGCATGATGGTGCACACCTGTGGT
CCCAGCTACTCAAGAGGCTGAAGGAGGATCACTTGAGTCCAGAAGGTCGAGGCTGCAGTGAGCTGTGATC
ATGCCACTGCACTCCAGCCTAAGTGACAGTGCGGTACCCTGTCTCAAAAAAAAAAAAAAAAAAAAAAAGA
GGTTGGAGCAGGAGGAAGCATAGGGGCGGGAACAGCCACCTCCTCCATGCCCTAGATTGTGAATTTATCG
GGCAGCCAACACATGTATGACACACTAGGCCCTGTATTACAGCTTGTTACGCATTTCATAAAAGGGATTT
TCATTAAGGAGA TAAT CTATTACTACCTACCTTAGTGGCTACTAGTATAAAACTATGACAGA TTTAGCAA
TTAAATGAAATACTGGCCTCCATCAAATAATCATAGTAACAAGAAGCAGCAGTTACCAGACATCTGATCC
CCTTCCCCCAAAATACCCAAATTCTTCATGGTTCTGCCCTTCTCTGTCCTTTCTGCTCCCCTTGCTCGCC
TGGGAAATGGAGGAAAGGCCTTCCCTCTCACACTGTCTTGGGATCTTGCTGAGAATTCAGACTGCTCGAA
ACAGTGACAAACCCCAGCCATCCAGTCATTCGTGGAGCACAATTTGGATGTGGCCCCAGGGGCATCTGTC
CCATTCAGAGAACCTTGGCAGTGCGATGGCCACTGTTCCCAGGCTTCAACCTCAGTGACCCCCCCCAACA
ACTCCCCATGGAGAGTCCCTGCCCAAAAAAGCTGTAGGATCCAAGGGGTGTCAATAGCTCGTTCCCGGCA
TCACCTACACACCACAAGCAGGTTTTAATGGAAGCAAGTTGCTCCACCAAATCCACAAAAGGGTAAAGTT
TGTGATTTTTCTTTATCATTGCGATCACCATCTGATACCGTAAGGAGTGCACTTGTTTGGAAGTTCTGAC
TTCTCTGATCTGTCTTGGTCGTTTGTGTTATAAAACCAAAGTTCTCTACAGACTTTATTTTTGTACAATA
TCATTTTGTAACTTTTTACAAATAAAAACTCATTTCTATTGC

A representative cDNA encoding isoform 1 is as follows.

(SEQ ID NO: 5)
caaatatgccagaggattatcctgatcagttcgatgatgtcatggatttta
ttcaagccaccattaaaagactgaaaaggtcaccagataaacaaatggcag
tgcttcctagaagagagcggaatcggcaggctgcagctgccaacccagaga
attccagaggaaaaggtcggagaggccagaggggcaaaaaccggggttgtg
tcttaactgcaatacatttaaatgtcactgacttgggtctgggctatgaaa
ccaaggaggaactgatttttaggtactgcagcggctcttgcgatgcagctg
agacaacgtacgacaaaatattgaaaaacttatccagaaatagaaggctgg
tgagtgacaaagtagggcaggcatgttgcagacccatcgcctttgatgatg
acctgtcgtttttagatgataacctggtttaccatattctaagaaagcatt
ccgctaaaaggtgtggatgtatctga

GenBank Accession No. L19063.1. gi:306761. See Science 1993, 260 (5111):1130-2.

The amino acid sequences of the various human GDNF isoforms are as follows

>gi |40549411 |ref |NP_954701.1| glial cell line-
derived neurotrophic factor isoform 2 pre-
proprotein [Homo sapiens]
(SEQ ID NO: 6)
MKLWDVVAVCLVLLHTASAFPLPAANMPEDYPDQFDDVMDFIQATIKRL
KRSPDKQMAVLPRRERNRQAAAANPENSRGKGRRGQRGKNRGCVLTAIH
LNVTDLGLGYETKEELIFRYCSGSCDAAETTYDKILKNLSRNRRLVSDK
VGQACCRPIAFDDDLSFLDDNLVYHILRKHSAKRCGCI
>gi |4503975 |ref |NP_000505.1| glial cell line-
derived neurotrophic factor isoform 1 pre-
proprotein [Homo sapiens]
(SEQ ID NO: 7)
MKLWDVVAVCLVLLHTASAFPLPAGKRPPEAPAEDRSLGRRRAPFALSS
DSNMPEDYPDQFDDVMDFIQATIKRLKRSPDKQMAVLPRRERNRQAAAA
NPENSRGKGRRGQRGKNRGCVLTAIHLNVTDLGLGYETKEELIFRYCSG
SCDAAETTYDKILKNLSRNRRLVSDKVGQACCRPIAFDDDLSFLDDNLV
YHILRKHSAKRCGCI
>gi |299473779 |ref |NP_001177397.1| glial cell
line-derived neurotrophic factor isoform 3
preproprotein [Homo sapiens]
(SEQ ID NO: 8)
MQSLPNSNGAAAGRDFKMKLWDVVAVCLVLLHTASAFPLPAGKRPPEAP
AEDRSLGRRRAPFALSSDSNMPEDYPDQFDDVMDFIQATIKRLKRSPDK
QMAVLPRRERNRQAAAANPENSRGKGRRGQRGKNRGCVLTAIHLNVTDL
GLGYETKEELIFRYCSGSCDAAETTYDKILKNLSRNRRLVSDKVGQACC
RPIAFDDDLSFLDDNLVYHILRKHSAKRCGCI
>gi |299473781 |ref |NP_001177398.1| glial cell
line-derived neurotrophic factor isoform 4
preproprotein [Homo sapiens]
(SEQ ID NO: 9)
MQSLPNSNGAAAGRDFKMKLWDVVAVCLVLLHTASAFPLPAANMPEDYP
DQFDDVMDFIQATIKRLKRSPDKQMAVLPRRERNRQAAAANPENSRGKG
RRGQRGKNRGCVLTAIHLNVTDLGLGYETKEELIFRYCSGSCDAAETTY
DKILKNLSRNRRLVSDKVGQACCRPIAFDDDLSFLDDNLVYHILRKHSA
KRCGCI

Like other growth factors GDNF has to be cleaved for secretion from the cell, and proteolytically processed for activation and also requires glycosaminoglycans for activation of specific signaling pathways. The GDNF amino acid sequence contains two potential glycosylation sites (discussed in greater detail below).

A multiple sequence alignment of the human GDNF isoforms is presented below. Signal sequences are depicted in bold. Signal peptides are aa 1-19, aa 1-19, and aa 1-36 for isoforms 1, 2 and 3, respectively. Mature peptides are depicted in italics. Mature peptides are aa 78-211, aa 52-185, and aa 95-228 for isoforms 1, 2 and 3, respectively. GDNF proteins have a key functional domain, termed the “transforming growth factor beta (TGF-β) like domain. TGF-P-like domains are aa 118-211, aa 92-185, and aa 135-228, for isoforms 1, 2 and 3, respectively. TGF-P-like domains are underlined.

| CLUSTAL 2.1 multiple sequence alignment (SEQ ID NOs: 6-9) | |
|---|---|
| isol | -----------------MKLWDWAVCLVLLHTAS AFPLPAGKRPPEAPAEDRSLGRRRA |
| iso3 | MQSLPNSNGAAAGRDFKMKLWDWAVCLVLLHTASAFPLPAGKRPPEAPAEDRSLGRRRA |
| iso2 | -----------------MKLWDWAVCLVLLHTAS AFPLPAAN----------------- |
| iso4 | MQSLPNSNGAAAGRDFKMKLWDWAVCLVLLHTASAFPLPAAN------------------ |
| | ***********************.: |
| isol | PFALSSDSNMPEDYPDQFDDVMDFIQATIKRLKRSPDKQMAVLPRRERNRQAAAANPENS |
| iso3 | PFALSSDSNMPEOYPOQFOOVMOFIQATIKRLKRSPDKQMAVLPRRERNRQAAAANPENS |
| iso2 | ---------MPEDYPDQFDDVMDF IQATIKRhKRSPDKQMA VLPRRERNRQAAAANPENS |
| iso4 | ---------MPEDYPDQFDDVMDF IQAT IKRLKRSPDKQMAVLPRRERNRQAAAANPENS |
| | ****************************************************** |
| isol | RGKGRRGQRGKNR GCVLTAIHLNVTDLGLGYETKEEL IFRYCSGSCDAAETTYDKILKNL |
| iso3 | RGKGRRGQRGKNR GCVLTAIHLNVTDLGLGYETKEEL IFRYCSGSCDAAETTYDKILKNL |
| iso2 | RGKGRRGQRGKNR GCVLTAIHLNVTDLGLGYETKEEL IFRYCSGSCDAAETTYDKILKNL |
| iso4 | RGKGRRGQRGKNR GCVLTAIHLNVTDLGLGYETKEEL IFRYCSGSCDAAETTYDKILKNL |
| | ************************************************************** |
| isol | SRNRRL VSDKVGQACCRP IAFDDDL SFLDDNL VYHILRKHSAKRCGCI |
| iso3 | SRNRRL VSDKVGQACCRP IAFDDDL SFLDDNL VYHILRKHSAKRCGCI |
| iso2 | SRNRRL VSDKVGQACCRP IAFDDDL SFLDDNL VYHILRKHSAKRCGCI |
| iso4 | SRNRRL VSDKVGQACCRP IAFDDDL SFLDDNL VYHILRKHSAKRCGCI |
| | **************************************************** |

As mentioned above, the GDNF gene encodes a highly conserved neurotrophic factor. GDNF orthologs share, for example, about 90-95% identity (or more), e.g., human and rat sharing 92% identity (10 amino acids (aa) are different) and human and mouse sharing 94% identity (8 amino acids are different) when comparing mature protein sequences; human and rat sharing 92% identity and human and mouse sharing 93% identity when comparing preproprotein sequences.

Pairwise alignments of human vs. mouse and human vs. rat preproproteins are presented below. The glycosylation sites are Asn (=N) 126 and Asn 162, and are indicated in bold. The skilled artisan will understand that a glycosylation motif is described as NX[ST] where X=any amino acid. The glycosylation motifs in, for example, human GDNF (isoform 1) are as follows aa126-128 (with N-linked glycosylation predicted to occur at N126, and at aa162-164 (with N-linked glycosylation predicted to occur at N162.) (See e.g., Lin et al., 1994, J. Neurochem, 63, 758-68; Trupp et al., 1995, J. Cell Biol, 130, 137-48).

| | | | |
|---|---|---|---|
| Hu | 1 | MKLWDWAVCLVLLHTASAFPLPAGKRPPEAPAEDRSLGRRRAPFALSSDSNMPEDYPDQ | 60 |
| | | MKLWDWAVCLVLLHTASAFPLPAGKR EAPAED SLG RR PFAL+SDSNMPEDYPDQ | |
| Mu | 1 | MKLWDWAVCLVLLHTASAFPLPAGKRLLEAPAEDHSLGHRRVPFALTSDSNMPEDYPDQ | 60 |
| Hu | 61 | FDDVMDFIQATIKRLKRSPDKQMAVLPRRERNRQAAAANPENSRGKGRRGQRGKNRGCVL | 120 |
| | | FDDVMDFIQATIKRLKRSPDKQ A LPRRERNRQAAAA+PENSRGKGRRGQRGKNRGCVL | |
| Mu | 61 | FDDVMDFIQATIKRLKRSPDKQAAALPRRERNRQAAAASPENSRGKGRRGQRGKNRGCVL | 120 |
| Hu | 121 | TAIHLNVTDLGLGYETKEELIFRYCSGSCDAAETTYDKILKNLSRNRRLVSDKVGQACCR | 180 |
| | | TAIHLNVTDLGLGYETKEELIFRYCSGSC++AET YDKILKNLSR+RRL SDKVGQACCR | |
| Mu | 121 | TAIHLNVTDLGLGYETKEELIFRYCSGSCESAETMYDKILKNLSRSRRLTSDKVGQACCR | 180 |
| Hu | 181 | PIAFDDDLSFLDDNLVYHILRKHSAKRCGCI | 211 |
| | | P+AFDDDLSFLDDNLVYHILRKHSAKRCGCI | |
| Mu | 181 | PVAFDDDLSFLDDNLVYHILRKHSAKRCGCI | 211 |
| Hu | 1 | MKLWDVVAVCLVLLHTASAFPLPAGKRPPEAPAEDRSLGRRRAPFALSSDSNMPEDYPDQ | 60 |
| | | MKLWDVVAVCLVLLHTASAFPLPAGKR EAPAED SLG RR PFAL+SDSNMPEDYPDQ | |
| Ra | 1 | MKLWDVVAVCLVLLHTASAFPLPAGKRLLEAPAEDHSLGHRRVPFALTSDSNMPEDYPDQ | 60 |
| Hu | 61 | FDDVMDFIQATIKRLKRSPDKOMAVLPRRERNRQAAAANPENSRGKGRRGQRGKNRGCVL | 120 |
| | | FDDVMDFIQATIKRLKRSPDKQ A LPRRERNRQAAAA+PENSRGKGRRGQRGKNRGCVL | |
| Ra | 61 | FDDVMDFIQATIKRLKRSPDKQAAALPRRERNRQAAAASPENSRGKGRRGQRGKNRGCVL | 120 |
| Hu | 121 | TAIHLNVTDLGLGYETKEELIFRYCSGSCDAAETTYDKILKNLSRNRRLVSDKVGQACCR | 180 |
| | | TAIHLNVTDLGLGYETKEELIFRYCSGSC+AAET YDKILKNLSR+RRL SDKVGQACCR | |
| Ra | 121 | TAIHLNVTDLGLGYETKEELIFRYCSGSCEAAETMYDKILKNLSRSRRLTSDKVGQACCR | 180 |
| Hu | 181 | PIAFDDDLSFLDDNLVYHILRKHSAKRCGCI | 211 |
| | | P+AFDDDL FLDD+LVYHILRKHSAKRCGCI | |
| Ra | 181 | PVAFDDDLWFLDDSLVYHILRKHSAKRCGCI | 211 |

Human, mouse and rat GDN sequence are set forth as SEQ ID NOs: 6, 10 and 11, respectively. The sequences appearing between aligned sequences above can be considered consensus sequences and are set forth as SEQ ID NOs: 12-13 (where no match between amino acids in aligned sequences can be depicted as X in a consensus sequence, X being one of the two mismatched residues, as depicted.

A multiple sequence alignment of human (isoform 1), mouse and rat GDNF orthologs (mature proteins) is presented below:

| | | | |
|---|---|---|---|
| Hu | 78 | SPDKOMAVLPRRERNRQAAAANPENSRGKGRRGQRGKNRGCVL | 120 |
| Mu | 78 | SPDKQAAALPRRERNRQAAAASPENSRGKGRRGQRGKNRGCVL | 120 |
| Ra | 78 | SPDKQAAALPRRERNRQAAAASPENSRGKGRRGQRGKNRGCVL | 120 |
| Hu | 121 | TAIHLNVTDLGLGYETKEELIFRYCSGSCDAAETTYDKILKNLSRNRRLVSDKVGOACCR | 180 |
| Mu | 121 | TAIHLNVTDLGLGYETKEELIFRYCSGSCESAETMYDKILKNLSRSRRLTSDKVGQACCR | 180 |
| Ra | 121 | TAIHLNVTDLGLGYETKEELIFRYCSGSCEAAETMYDKILKNLSRSRRLTSDKVGQACCR | 180 |
| Hu | 181 | PXAFDDDLSFLDDNLVYHILRKHSAKRCGCI | 211 |
| Mu | 181 | PVAFDDDLSFLDDNLVYHILRKHSAKRCGCI | 211 |
| Ra | 181 | PVAFDDDLWFLDDSLVYHILRKHSAKRCGCI | 211 |

The above sequences are set forth as amino acids 78-21 1 of SEQ ID NOs: 6, 10 and 11 respectively.

Mature protein sequences are also envisioned in which a methionine (Met; M) precedes the first amino acid of the mature sequence. The M can be added for recombinant protein expression of mature proteins, and is encoded in engineered cDNA expression systems. However, the skilled artisan will appreciate that there are also expression systems which allow the cleavage of the N-terminal M, as it can induce autoimmune reactions or changes in activity of the expressed, mature protein. For example see Nakagawal et al. 1987, Nature Biotech 5, 824-827; Fernandez-San Millan et al., 2007, J Biotechnol. 20; 127(4):593-604; U.S. Pat. No. 4,870,017.

GDNF sequences are as described above and additional information on said sequences can be found in the GenBank references indicated by the referenced GenBank/gi reference numbers. GDNF sequences are also described in Science. 1993 May 21; 260(5111):1130-2; and in WO 93/06116 and U.S. Pat. No. 7,226,758B1. Truncated forms are further described in US20040127419(A1). Mutations of GDNF are described, for example in Eketjall et al. 1999, EMBO 8, 5901-5910. The GDNF protein is further disclosed in, e.g., U.S. Pat. No. 6,362,319 and European Patent No. 0 610 254, and a truncated form of GDNF in U.S. Pat. No. 6,184,200 and European Patent No. 0 920 448.

Exemplary aspects of the invention can further include modified (e.g., recombinantly-modified) forms of GDNF, or of biologically active fragments thereof. In one embodiment, the method of the invention feature the use of fusion proteins of GDNF, for example, fusion proteins including serum albumin or a biologically active fragment thereof, e.g., human serum albumin or a biologically active fragment thereof.) Further exemplary aspects of the invention feature pegylated GDNF proteins, glycan-modified GDNF proteins (e.g, having N-glycan integrated within the protein) and/or polymer-conjugated GDNF (e.g., polymers consisting of a polystyrene backbone with side chains of trehalose.)

Preferred aspects of the invention feature GDNF polypeptides, GDNF homologs (e.g., GDNF orthologs) and/or biologically active portions (i.e., bioactive fragments) of GDNF polypeptides. In one embodiment, GDNF polypeptides can be isolated from cells or tissue sources by an appropriate purification scheme using standard protein purification techniques. GDNF polypeptide can be further derived from said isolated polypeptides using standard enzymatic techniques. In another embodiment, GDNF polypeptides or bioactive fragments thereof are produced by recombinant DNA techniques. Alternative to recombinant expression, GDNF polypeptides or bioactive fragments thereof can be synthesized chemically using standard peptide synthesis techniques.

Polypeptides of the invention are preferably “isolated” or “purified”. The terms “isolated” and “purified” are used interchangeably herein. “Isolated” or “purified” means that the protein or polypeptide is substantially free of cellular material or other contaminating proteins from the cell or tissue source from which the polypeptide is derived, substantially free of other protein fragments, for example, non-desired fragments in a digestion mixture, or substantially free from chemical precursors or other chemicals when chemically synthesized. The language “substantially free of cellular material” includes preparations in which the polypeptide is separated from other components of the cells from which it is isolated or recombinantly produced. In one embodiment, the language “substantially free of cellular material” includes preparations of polypeptide having less than about 30% (by dry weight) of non-GDNF polypeptide (also referred to herein as a “contaminating protein”), more preferably less than about 20% of non-GDNF polypeptide, still more preferably less than about 10% of non-GDNF polypeptide, and most preferably less than about 5% non-GDNF polypeptide. When the polypeptide or protein is recombinantly produced, it is also preferably substantially free of culture medium, i.e., culture medium represents less than about 20%, more preferably less than about 10%, and most preferably less than about 5% of the volume of the polypeptide preparation. When the polypeptide or protein is produced by, for example. chemical or enzymatic processing from isolated or purified GDNF protein, the preparation is preferably free of enzyme reaction components or chemical reaction components and is free of non-desired GDNF forms, e.g. aggregates, or GDNF fragments, i.e., the desired polypeptide represents at least 75% (by dry weight) of the preparation, preferably at least 80%, more preferably at least 85%, and even more preferably at least 90%, 95%, 99% or more or the preparation.

The language “substantially free of chemical precursors or other chemicals” includes preparations of polypeptide in which the polypeptide is separated from chemical precursors or other chemicals which are involved in the synthesis of the polypeptide. In one embodiment, the language “substantially free of chemical precursors or other chemicals” includes preparations having less than about 30% (by dry weight) of chemical precursors or reagents, more preferably less than about 20% chemical precursors or reagents, still more preferably less than about 10% chemical precursors or reagents, and most preferably less than about 5% chemical precursors or reagents.

Bioactive fragments of GDNF polypeptides include polypeptides comprising amino acid sequences sufficiently identical to or derived from the amino acid sequence of the GDNF protein, respectively, which include less amino acids than the full length protein, and exhibit at least one biological activity of the full-length protein. Typically, biologically active portions comprise a domain or motif with at least one activity of the full-length protein. A biologically active portion of a GDNF polypeptide can be a polypeptide which is, for example, 10-20, 20-30, 30-40, 40-50, 50-60, 60-70, 70-80, 80-90, 90-100, 100-120, 120-140, 140-160, 160-200, or more amino acids in length. In a preferred embodiment, a bioactive portion of a GDNF protein comprises a TGFP-like domain. Moreover, other biologically active portions, in which other regions of the protein are deleted, can be prepared by recombinant techniques and evaluated for one or more of the functional activities of a native GDNF protein. Mutants of GDNF can also be utilized as assay reagents or therapeutic or pharmaceutical agents, for example, mutants having reduced, enhanced or otherwise altered biological properties identified according to one of the activity assays described herein.

As defined herein, a GDNF polypeptide of the invention includes polypeptides having the amino acid sequences set forth above, as well as homologs and/or orthologs of said polypeptides, i.e. polypeptides having sufficient sequence identity to function in the same manner as the described polypeptides. To determine the percent identity of two amino acid sequences (or of two nucleotide or amino acid sequences), the sequences are aligned for optimal comparison purposes {e.g., gaps can be introduced in the first sequence or second sequence for optimal alignment). The amino acid residues or nucleotides at corresponding amino acid positions or nucleotide positions are then compared. When a position in the first sequence is occupied by the same residue as the corresponding position in the second sequence, then the molecules are identical at that position. The percent identity between the two sequences is a function of the number of identical positions shared by the sequences {i.e., % homology=#of identical positions/total #of positions×100), optionally penalizing the score for the number of gaps introduced and/or length of gaps introduced.

The comparison of sequences and determination of percent identity between two sequences can be accomplished using a mathematical algorithm. In one embodiment, the alignment generated over a certain portion of the sequence aligned having sufficient identity but not over portions having low degree of identity (i.e., a local alignment). A preferred, non-limiting example of a local alignment algorithm utilized for the comparison of sequences is the algorithm of Karlin and Altschul (1990) Proc. Natl. Acad. Sci. USA 87:2264-68, modified as in Karlin and Altschul (1993) Proc. Natl. Acad. Sci. USA 90:5873-77. Such an algorithm is incorporated into the BLAST programs (version 2.0) of Altschul, et al. (1990) J. Mol. Biol. 215:403-10. BLAST alignments can be generated and percent identity calculated using BLAST protein searches (e.g., the XBLAST program) using GDNF protein, or a portion thereof as a query, score=50, wordlength=3.

In another embodiment, the alignment is optimized by introducing appropriate gaps and percent identity is determined over the length of the aligned sequences {i.e., a gapped alignment). To obtain gapped alignments for comparison purposes, Gapped BLAST can be utilized as described in Altschul et al., (1997) Nucleic Acids Research 25(17):3389-3402. In another embodiment, the alignment is optimized by introducing appropriate gaps and percent identity is determined over the entire length of the sequences aligned {i.e., a global alignment). A preferred, non-limiting example of a mathematical algorithm utilized for the global comparison of sequences is the algorithm of Myers and Miller, CABIOS (1989). Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

A GDNF bioactive fragment is any fragment of GDNF having sufficient size and structure to carry out at least one activity (e.g., biological activity) of the corresponding full-length GDNF protein. Exemplary bioactive fragments include, but are not limited to, enzymatic domains, protein binding and/or interaction domains, receptor binding domains, and the like. Preferred bioactive fragments include regions or domains comprising a TGFP-like domain, as defined herein.

The GDNF protein or GDNF bioactive fragment, may be detectably labeled. As defined herein, a protein or protein fragment which is “detectably labeled” is on which has been modified to include a component detectable by standard laboratory means, e.g., the protein has been radioactively labeled, chromogenically labeled, or fluorescently labeled. Labeling can be direct, i.e., protein is modified to directly contain the detectable label, or can be indirect, e.g., the protein is modified to include a component with which the detectable label interacts. Furthermore, in other embodiments, the activity of a GDNF protein or GDNF bioactive fragment may be compared to an appropriate control.

In preferred embodiments, a GDNF polypeptide or homolog or bioactive fragment thereof includes at least a TGFp-like, as defined herein. The TGFp-like domain is a domain conserved among most, if not all TGFP family members. To identify the presence of a TGFp-like domain in an GDNF polypeptide, the amino acid sequence of the polypeptide can be searched against a database of conserved protein domains (e.g., the CD database at the NCBI) using the default parameters (Marchler-Bauer A et al. (2013), “COO: conserved domains and protein three-dimensional structure.” Nucleic Acids Res. 41(D1):D384-52.). NCBI Conserved Domains Database Accession number pfam00019 sets forth a conserved TGFp-like domain amino acid sequence.

In exemplary embodiments, a TGFp-like domain includes about 90-110 amino acid residues, e.g., about 90-100 or 91, 92, 93, 94, 95, 96, 97, 98, 99 or 100 amino acid residues). In exemplary embodiments, a domain identification score of 60 is a suitable threshold score for determining the presence of the domain. For example, searches using the amino acid sequences of human GDNF (isoform 1), mouse GDNF and rat GDNF were performed against the CD database resulting in the identification of a TGFp-like domain at amino acids 118-211 in each protein.

In preferred embodiments, a GDNF composition is a mature human GDNF protein consisting of 134 amino acids, i.e., amino acids 78-211 of SEQ ID NO:6. This sequence contains two putative N-glycosylation sites as well as seven conserved cysteines in the same relative spacing as the other members of the TGF-beta protein family (Lin et al. 1993, Science, 260: 1130-1132: Eigenbrot and Gerber, 1997, Nat Struct Biol, 4:435-438; Chang et al. 2002. Endocri Rev, 23:787-823). Biologically active mature GDNF dimer is formed by a covalent disulfide bond between the unpaired cysteines in the monomers (Eigenbrot and Gerber, 1997, Nat Struct Biol, 4:435-438).

In other exemplary embodiments, a GDNF protein for use in the methods of the invention can be a variant GDNF protein (or polypeptide), e.g., a variant having at least 90% or at least 95% or more identity. Preferred are biologically active variant GDNF polypeptides. As used herein, the phrase “biologically active variant GDNF polypeptide” refers to a GDNF polypeptide that, when dimerized, binds to a temary complex containing GFRal and RET. Any method for detecting binding to this complex can be used to evaluate the biological activity a variant GDNF polypeptide. Exemplary assays for detecting the ternary complex-binding ability of a variant GDNF polypeptide are described in WOOO/01815. Variant GDNF polypeptides can also be assayed or tested for their ability to trigger a GDNF signaling cascade. For example, a kinase receptor activation (KIRA) assay can be used to assess the ability of a variant GDNF polypeptide to induce RET autophosphorylation (see e.g., Sadick et al., 1996, Anal. Biochem., 235(2): 207) or assays can be performed to detect expression of downstream targets of a GDNF signaling cascade, e.g., increased expression of ret orfgfr2.

### III. Wound Healing

The body's response to skin injury is focused on rapid wound closure, restraining invasion of microorganisms, and preventing excessive fluid loss (Singer AJ, et al., N Engl J Med 341:738-46, 1999; Aarabi S, et al., PLoS Med 4:e234, 2007; Gurtner GC, et al., Nature 453:314-21, 2008; Mustoe T., Am. J Surg 187:65S-70S, 2004).

An increased understanding of the molecular mechanisms that regulate the various events of wound healing has laid the foundation for therapeutic interventions attempting to improve the healing outcome. The cell-cell and cell-matrix interactions are fundamental for successful wound healing, and growth factors and cytokines maintain the balance of signals that regulate cellular migration, proliferation, and adhesion to a large extent. Freedberg IM, et al., J Invest Dermatol 116:633-40, 2001: Hantash BM, et al., Front Biosci 13:51-61, 2008: Werner S. et al, Physiol Rev 83:835-70, 2003; Barrientos S, et al., Wound Repair Regen 16:585-601, 2008. Malfunction leads to a prolonged healing time or complete failure to heal and may result in a chronic wound. The wound fluid from chronic wounds has an increased concentration of proinflammatory cytokines in comparison with wound fluid from acute wounds. Bennett NT, et al., Am J Surg 166:74-81, 1993; Robson MC, et al., Arch Surg 135:773-7, 2000. By contrast, there is a decreased concentration of growth factors in chronic wounds with high protease activity and decreased levels of natural protease inhibitors. Nwomeh BC, et al., Clin Plast Surg 25:341-56, 1998; Mast BA, et al, WoundRepair Regen 4:411-20, 1996; Tarnuzzer RW, et al., WoundRepair Regen 4:321-5, 1996. This deficiency in growth factors can be attributable to decreased production or secretion, more rapid breakdown, and, as is the case in venous stasis ulcers, binding to in macromolecules, making them nonfunctional. Robson MC, et al., Arch Surg 135:773-7, 2000; Falange V, et al., Lancer 341:1006-8, 1993.

Glial cell line-derived neurotrophic factor (GDNF), neurturin (NTN), and their receptors, GDNF family receptor a-1 (GFRa-1) and GDNF family receptor a-2 (GFRa-2), are critically important for development in systems such as kidney and nervous system. Moreover, Gdnf has been shown to be expressed in embryonic skin where Gdnf mRNA is detected in both epithelial and mesenchymal components (Hellmich et al. 1996, Mech Dev 54, 95-105). However, the role of these factors (e.g., GDNF) in skin biology, and in particular, wound healing, is as yet not fully understood. Knowledge of the role of this important factor in skin biology would be helpful in understanding not only the various normal wound-healing events but also those occurring under distinct pathological conditions, for example, conditions in which wound healing is impaired, e.g., pathological conditions such as diabetes. In addition, development of effective novel therapies for wound healing can based, at least in part, on this better understand of the effect of GDNF on the total wound-healing process. This approach facilitates the development of new products with potential applications in wound healing and other area of skin biology.

In humans, and more widely in all mammalian species, the wound-healing process can be subdivided into three consecutive and overlapping phases: inflammation, tissue formation, and matrix formation and remodeling. Rodero, M.P., et al., Int. J. Clin. Exp. Pathol. 3:643653, 2010. The transition from one phase to another depends on the maturation and differentiation of the main cell populations involved, among which keratinocytes, fibroblasts, neutrophils, and macrophages play the main roles. Rodero, M.P., et al., Int. J. Chin. Exp. Pathol. 3:643653, 2010; Leibovich. S.J., et αI., Aη. J. Pathol. 78:71-100, 1975; Deonarine, K., et al., J. Transl. Med. 5:11. 2011; Becker. D.L., et al., Biochim. Biophys Acta 1818:2068-2075. 2011. Recent observations show that stem cells have an unclear but likely major role in response to cutaneous injury, Lanza R., Handbook of Stem Cells. Academic Press, 2004, as well as the evidence for the roles of M1 and M2 macrophage, and that of T cells. Gilliver, S.C., et al., Exp. Dermatol. 20:1-6, 2011; Sindrilaru, A., et al. J. Clin. Invest. 121:985-997, 2011.

Initial stages of wound healing involve the formation of a blood clot and inflammation. The inflammatory response is followed by proliferation and migration of dermal and epidermal cells, and matrix synthesis, in order to fill the wound gap and reestablish the skin barrier (Cotran, et al., 1999; Hackam, D.J., et al. Surg Infect 3 (Suppl 1), S23-5 (2002); Harding, K.G., et al. Int Wound J 2. 364-8 (2005)). Finally, tissue remodeling and differentiation enable full recovery of the skin tissue and restoration of skin aesthetics (Hackam, D.J., et al. Surg Infect 3 (Suppl 1), S23-5 (2002); Diegelmann, R.F., et al. Front Biosci 9, 283-9 (2004). The consensus in the literature is that the stepwise process of wound healing first strives toward immediate filling of the gap, followed by re-epithelialization and reestablishment of the skin barrier (Yamaguchi, Y., et al. J Dermatol 28, 521-34, (2001)).

The early response is activated immediately after injury, resulting in the inflammatory phase (first stage) of wound healing. Grose R, et al., Mol Biotechnol 28:147-66, 2004. After hemostasis a fibrin clot is formed, which later serves as a scaffold for infiltrating cells. In addition, neutrophils and monocytes are recruited to the wound in response to trauma and bacterial contamination. Martin P, et al., Trends Cell Biol 15:599-607, 2005. In detail, the first event occurring after injury is the formation of a blood clot; several cells are involved in the blood plug: platelets, and red and white blood cells. With the action of fibrin fibers, the clot is stabilized and then “invaded” by several infiltrating cells, such as neutrophils, macrophages, mastocytes, platelets, and, possibly, by bacteria and toxins, which are counteracted by host-generated H2O2. Neutrophils massively infiltrating the wound during the first 24 hours postinjury are attracted by numerous inflammatory cytokines produced by activated platelets, endothelial cells, as well as by degradation products from pathogens. Macrophages massively infiltrating the wound two days postinjury produce intense phagocytic activity. Mosser, D.M., et al., Nature Rev. Immunol. 8:958-969, 2008.

The second stage of wound repair (tissue formation) occurs approximately 2 to 10 days after the injury and is characterized by proliferation and migration of different cell types. Keratinocytes migrate over the wound bed while fibroblasts and macrophages replace the fibrin clot with granulation tissue. Werner S, et al., J Invest Dermatol 27:998-1008, 2007. The newly formed immature dermis is neovascularized, and the keratinocytes behind the leading edge proliferate and differentiate to restore the barrier function of the epidermis. In detail, after two to three days, the second phase lasts about two weeks and is characterized by neo-angiogenesis and granulation. During the re-epithelialization process, keratinocytes from the wound edges migrate over the wound bed at the interface between the wound dermis and the fibrin clot. This migration is facilitated by the production of specific proteases, such as collagenase by the epidermal cells to degrade the extracellular matrix. Activated fibroblasts also migrate to the wound bed and form, with the macrophages, granulation tissue. Intense angiogenesis, allowing the supply of oxygen and nutrients necessary for the healing process, also occurs within the tissue. Both growth factors and reactive oxygen species (ROS) produced by the granulation tissue will favor proliferation and differentiation of epithelial cells, restoring epithelial barrier integrity

Tissue remodeling, the third stage of wound repair, begins 2 to 3 weeks after injury and lasts for 1 year or more. The type III collagen that is deposited in the initial stages of wound healing is slowly replaced by type I collagen, thereby forming the mature dermis. Lowom HN 3^{rd}, et al, J Pediatr Surg 34:218-23, 1999. In detail, The last stage of the wound healing process consists in a gradual involution of the granulation tissue and dermal regeneration. This step is associated with apoptosis of myofibroblasts. endothelial cells, and macrophages. The remaining tissue is therefore composed mostly of extracellular matrix proteins, essentially collagen type III that will be remodeled by metalloproteinase produced by epidermal cells, endothelial cells, fibroblasts, and the macrophages remaining in the scar and then replaced by collagen type I. Singer, A.J., et al. N. Engl. J. Med. 341:738-746, 1999.

### IV. Hair Growth

As is the case for wound healing, the role of Glial cell line-derived neurotrophic factor (GDNF) and its receptors in hair growth control, is as yet not fully understood. As mentioned above, Gdnfhas been shown to be expresses in embryonic skin where Gdnf mRNA is detected in both epithelial and mesenchymal components (Hellmich et al. 1996, Mech Dev 54, 95-105). Gdnfhas also been shown to be expressed during human hair follicle development (Adly et al. 2008 J Am Acad Dermatol, 58:238-250) and has further been shown to be expressed in adult mice in hair follicles during the anagen to catagen transition phase (Botchkareva et al. 2000, Am J Pathol 156,1041-1053). These gene expression patterns suggest a role for GDNF in the hair follicle development cycle. The instant inventors discovered that in a transgenic mouse model, when Gdnf is over-expressed under the Cathespin L promoter, it affects the hair follicle growth in mice. Further analysis suggested that the effect of GDNF on the hair follicles in these mice was possibly due to expression of Gdnf in cells expressing endogenous Cathespin L gene, cells of the outer and inner root sheath and is essential for regular hair follicle morphogenesis and cycling.

Hair grows in cycles of various phases: anagen is the growth phase; catagen is the involuting or regressing phase (also termed “apoptosis-driven regression”): and telogen, the resting or quiescent phase (see e.g., Stenn and Paus (2001) “Controls of Hair Follicle Cycling”. Physiological Reviews 81 (1): 449-494 and Paus et al., “The biology of hair follicles”, NEJM 1999, 341:491-497). The time these phases last varies from person to person. Different hair color and follicle shape affects the timings of these phases, anagen phase, 2-3 years (e.g., approximately 3 years, occasionally much longer); catagen phase, 2-3 weeks; and telogen phase, around 3 months.

Each phase has several morphologically and histologically distinguishable sub-phases. Prior to the start of cycling is a phase of follicular morphogenesis (formation of the follicle). There is also a shedding phase, or exogen, that is independent of anagen and telogen in which one of several hair that might arise from a single follicle exits. Normally up to 90% of the hair follicles are in anagen phase while, 5-15% (or 10-14%) are in telogen and 1-2% in catagen. The cycle's length can vary depending on location on different parts of the body.

Anagen is the active growth phase of hair follicles. The root of the hair are dividing rapidly, adding to the hair shaft. During this phase the hair grows about 1 cm every 28 days. Scalp hair stays in this active phase of growth for 2-7 years. The amount of time the hair follicle stays in the anagen phase is genetically determined. At the end of the anagen phase an unknown signal causes the follicle to go into the catagen phase.

The catagen phase is a short transition stage that occurs at the end of the anagen phase. It signals the end of the active growth of a hair. This phase lasts for about 2-3 weeks while the hair converts to a club hair. A club hair is formed during the catagen phase when the part of the hair follicle in contact with the lower portion of the hair becomes attached to the hair shaft. This process cuts the hair off from its blood supply and from the cells that produce new hair. When a club hair is completely formed, about a 2 week process, the hair follicle enters the telogen phase.

The telogen phase is the resting phase of the hair follicle. During telogen, the resting hair remains in the follicle until it is pushed out by growth of a new anagen hair. In most people, 5-15% of the hair on the scalp is in telogen at any given time. Shedding does not occur until the new anagen hairs begin to grow. The emerging hairs help to force the resting hairs out of the follicle. Recent evidence suggests that the mechanism of shedding of a telogen hair is an active process that may occur independent of the emerging anagen hair. When the body is subjected to extreme stress, as much as 70 percent of hair can prematurely enter a phase of rest, called the telogen phase. This hair begins to fall, causing a noticeable Joss of hair. This condition is called telogen effluvium (see below). Telogen effluvium is a form of nonscarring alopecia characterized by diffuse hair shedding, often with an acute onset. Telogen effluvium can affect hair on all parts of the body, but, generally, only loss of scalp hair is symptomatic. Understanding the pathophysiology of telogen effluvium requires knowledge of the hair growth cycle (as detailed herein.) The club hair is the final product of a hair follicle in the telogen stage, and is a dead, fully keratinized hair. Fifty to one-hundred club hair are shed daily from a normal scalp.

The symptom of both acute and chronic telogen effluvium is increased hair shedding and diffuse hair loss from the entire scalp. Acute telogen effluvium is defined as hair shedding lasting less than 6 months. Patients usually only complain that their hair is falling out at an increased rate or that the remaining hair feels less dense. Causes for telogen effluvium and acute hair shedding can be physiologic stress, papulosquamous diseases of the scalp such as psoriasis and seborrheic dermatitis, allergic contact dermatitis, immunizations, severe infections (HIV), acute illness such as febrile illness, major surgery and severe trauma as well as chronic illness such as malignancy, particularly lymphoproliferative malignancy, systemic lupus erythematosus, end-stage renal disease, or liver disease, hormonal changes such as pregnancy and delivery (can affect both mother and child), hypothyroidism, discontinuation of estrogen-containing medications; changes in diet like crash dieting, anorexia, low protein intake, and chronic iron deficiency, heavy metals such as selenium, arsenic, and thallium. Acute telogen effluvium can occur in either sex, but because hormonal changes in the postpartum period are a common cause of telogen effluvium, women may have a greater tendency to experience this condition. Patients with acute telogen effluvium usually complain of relatively sudden onset of hair loss. If greater than 25% of extracted hairs are in telogen, the diagnosis of telogen effluvium is confirmed. However, each patient's scalp hair has an individual characteristic growth cycle. There are patients who have a very long anagen phase and a small proportion of hair in telogen at any given time. Telogen effluvium can be caused by medications, such as beta-blockers, anticoagulants, retinoids (including excess vitamin A), propylthiouracil (induces hypothyroidism), carbamazepine, and immunizations.

### V. Methods of Treatment

The instant invention features the use of GDNF for treatment in cases where wound healing and/or hair growth is desired. In one embodiment, the invention features a method of promoting cutaneous wound healing in a subject, which includes the step of administering at a wound site on the subject a composition comprising a therapeutically effective dose of GDNF, or a biologically active fragment thereof, and (optionally) repeating the administration for a time period sufficient to promote said cutaneous wound healing.

As defined herein, the phrase “cutaneous wound healing” refers to wound healing of the skin, in particular, the skin of a mammal. “Cutaneous wound healing” is also referred to in the art as “dermal wound healing.” Cutaneous wound healing is quite distinct from, for example, corneal wound healing. For example, the phases and sequence of events occurring in these type of wound healings differ. Moreover, the goals of these types of wound healing differ, for example, in the desired endpoint. Notably, one of the most crucial aspects of corneal wound healing is how the healing process aims to minimize end results such as vascularization and scar formation (which would have serious visual consequences). By contrast, such processes are significant desired end results of wound healing in other parts of the body, in particular, vascularization.

Administration is preferably topical administration but can also be achieved by injection of GDNF compositions of the invention at the wound site. In preferred embodiments, the wound site is external, e.g., on or in the skin of the subject. In exemplary embodiments, the wound site is an incision, a laceration, an abrasion, a puncture wound, a penetration wound, a surgical wound, an ulceration, a bum, a contusion, a hematoma, or a crush injury. In the case of topical administration, a GDNF composition of the invention can further include at least one anti-inflammatory agent. Alternatively, a GDNF composition of the invention can further include at least one antibiotic. Alternatively, a GDNF composition of the invention can further include at least one other wound healing-promoting agent.

In exemplary aspects of the invention, administration of GDNF composition is repeated for a time at least sufficient to promote filling and re-epithelialization of a wound site. In preferred aspects of the invention, administration of GDNF composition is repeated for a time at least sufficient to reestablish a skin barrier at the wound site.

In exemplary aspects, formulations of GDNF can contain at least about 1 ng/ml and up to about 10 μg/ml (e.g., for cosmetic applications), at least about 10 μg/ml and up to about 10 μg mg/ml or even 1 mg/ml (e.g., for wound healing and/or hair growth applications). In other exemplary aspects, a therapeutically effective dose consists of an amount of GDNF administered in a set period of time (e.g., daily), and routinely repeated over time (e.g., over weeks or months) to get the desired therapeutic effect. For example, GDNF compositions can be used at the concentration recited above and administered in a therapeutically effective dose (e.g., 1-100 ng daily, 100 ng to 1 μg daily, 1-50, 50-100, or 100-500 μg daily, 100 or 500 μg, up to 1 mg daily, 1-10 mg daily, 10-20 mg daily, 20-30 mg daily, 30-40 mg daily, or more. In some embodiments, a GDNF composition is administered daily. In other embodiments, a GDNF composition is administered multiple times a day e.g., twice or three times daily. The skilled artisan will readily appreciate that doses can be significantly lower if the compositions are to be administered via a controlled release system or formulation. For example, doses in the ng/ml or even pg/ml range are possible in the case of controlled release systems or formulations.

In other aspects, the invention features methods for promoting hair growth on a subject which includes the step of administering at site of desired hair growth on the subject (e.g., at a skin site where hair follicles grow) (e.g., on a scalp) a composition comprising a pharmaceutically effective dose of isolated GDNF, or a biologically active fragment thereof, and repeating the administration for a time period sufficient to promote said hair growth on said subject.

As a measure of efficacy, a pharmaceutically effective dose is a dose sufficient to promote a 5%, 10%. 15%, 20%, 25% increase in hair follicle number (or follicular units) over a period of several weeks to several months. Preferably, a pharmaceutically effective dose is a dose sufficient to promote a 10% increase in hair follicle number (or follicular units) over a period of several weeks to several months. Even more preferably, a pharmaceutically effective dose is a dose sufficient to promote a 50% (or more) increase in hair follicle number (or follicular units) over a period of several weeks to several months. In exemplary embodiments, the method of promoting hair growth involves administration of a GDNF composition via topical contacting at a skin site containing hair follicles (or a skin site that normally contains hair follicles). A skin site for hair growth can be virtually anywhere on the body except, for example, the soles of the feet and the palms of the hands, the lips, and the eyelids, apart from eyelashes.

As an alternative, efficacy of treatment can be monitored according to any art-recognized means for evaluating hair loss in a subject. Without being bound in theory, it is proposed that the methods of the invention can reduce hair shedding, for example, to a normal level. Exemplary non-invasive methods for monitoring hair loss include daily hair counts, standardized wash test, and the like (e.g., questionnaires, 60-s hair count, global photographs, dermoscopy, hair weight, contrasting felt examination, hair feathering test, phototrichogram and TrichoScan), which are good methods for primary evaluation of the patient and to get an approximate assessment of the amount of shedding. While not preferred, semi-invasive methods, e.g., Trichogram and unit area trichogram (UAT), and/or invasive methods, e.g., scalp biopsy, can also be used to measure hair loss (and, indirectly, hair growth.). For a detailed description of these procedures, see e.g., Dhurat, R. and Saraogi, P. 2009 Int J Trichology 1(2): 108-119.

As a measure of efficacy, a pharmaceutically effective dose is a dose sufficient to promote a 5%, 10%. 15%, 20%, 25% decrease in hair loss over a period of several weeks to several months. Preferably, a pharmaceutically effective dose is a dose sufficient to promote a 10% decrease in hair loss over a period of several weeks to several months. Even more preferably, a pharmaceutically effective dose is a dose sufficient to promote a 50% (or more) decrease in hair loss over a period of several weeks to several months.

In exemplary embodiments, the hair growth promoting methods of the invention are used to treat androgenetic alopecia (AGA), also known as male pattern baldness or female pattern baldness. In other embodiments, the hair growth promoting methods of the invention are used to treat autoimmune alopecia. This disease interferes with the hair growth cycle by causing a follicle to prematurely leave the anagen, or active growth, phase and enter the resting, or telogen phase. The hair growth in the affected follicles is lessened or stopped completely.

In other embodiments, the hair growth promoting methods of the invention are used to treat hair shedding. In other embodiments, the hair growth promoting methods of the invention are used to treat hair thinning. In yet other embodiments, the methods of the invention are used to treat acute or chronic telogen effluvium. In still other embodiments, the methods of the invention are used, generally, to treat scalp hair loss, hair thinning or baldness (poor hair thickness, poor hair growth). In yet another embodiment, the methods of the invention are used to extend the life or otherwise improve the condition of hair implants.

In other embodiments, the methods of the invention are used to treat iatrogenically-induced hair loss, for example, scalp hair and/or eye brow hair loss in chemotherapy patients. As used herein, the phrase “iatrogenically-induced” refers to a condition or disease state caused by a physician, surgeon, or other administering professional or by a medical or surgical treatment (e.g., pharmaceutical treatment, chemotherapeutic treatment, radiation treatment) or a diagnostic procedure.

In some embodiments, the GDNF compositions of the invention can be used in combination with one or more additional agents, e.g., art-recognized agents that promote wounds healing or hair growth and/or reduce hair loss. For example, the GDNF compositions of the invention might be used in combination, or in a combination therapy, with any one (or more than one) of the following agents: Combination platelet-derived growth factor (PDGF), interleukin-1 (IL1), nerve growth factor (NGF) or proNGF, keratinocyte growth factor (KGF), thymic peptides of the families of thymulin, thymosin alpha-1 and thymosin beta-4 (see e.g., US20110281802A1). Combination treatment can include, in other embodiments, coincident oral treatment with, for example, vitamins; combination of vitamin B1, vitamin B6, vitamin B12, folic acid, magnesium glycinate, L-cysteine, biotin, ferric glycinate, Polygonum multiflorum, and/or Emblica officinalis (sec e.g., US20130017285); insulin, insulin-like growth factor (IGF) (see e.g. US20100172865; Yoon et al. 2011, PLoS ONE 6(12): e28474), and/or polyamines (see e.g., Ramot et al. 2011. PLoS ONE 6(7): e22564). In some embodiments, the GDNF compositions of the invention can be used in combination with an electrical stimulus or mechanical stimulus (see e.g., Yoon et al. 2011, PLoS ONE.)

Without being bound in theory, it is also believed that GDNF, or a biologically active fragment thereof, can be administered to subjects via a gene therapy approach, for example, in cases where healing of chronic wounds is desired. GDNF-encoding nucleic acids can be engineered into appropriate expression vectors and targeted to areas requiring continued supply of expressed GDNF. Expression vectors can be engineered to encode GDNF, or a biologically active fragment thereof, in the context of a fusion protein, for example, fuse with a receptor targeting means for achieving entry into cells of the skin. Also, vectors can be formulated with agents that promote entry of nucleic acid molecules into skin.

### VI. Other Uses

### Chronic Wound Healing

The instant invention also features the use of GDNF for treatment in cases where a subject suffers from chronic nonhealing wounds. Experts debate about the time for closure that defines a chronic nonhealing wound as compared to that required for closure of an acute wound. Dealcy. C. 3rd ed. Blackwell Publishing Ltd., 2005; Whitney, J.D., Nurs. Clin. North Am. 40:191-205, 2005; Bryant, RA., et al. Acute and Chronic Wounds, Current Management Concepts. 4th ed. Mosby, 2011. It has been stated that “acute wounds generally follow trauma or inflammation and usually heal within six weeks.” Kumar, S., et al., Surgery 26:43-47, 2008. Chronic wounds (in addition to failing to heal after six weeks) have characteristic pathological associations that inhibit or delay the healing process. Jones, K.R., et al., Adv. Skin Wound Care 20:591-600, 2007.

Chronic wound healing is mainly sustained by chronic inflammation, which without appropriate therapy tends to worsen. The basic reasons are not necessarily old age but rather hypertension and atherosclerosis, which can lead to ischemia, diabetes, and venous insufficiency. Common pathogenetic causes are local tissue hypoxia, edema, abundant bacterial colonization, and, possibly, repeated ischemia-reperfusion injuries. The surface area of a nonhealing wound tends to widen and shows fibrin deposition, necrotic areas, and a few islands of granulation tissue. It is estimated that, in the industrialized world, 1-1.5% of the population experience problems related to recovering proper skin function. The problem is particularly prominent in elderly and diabetic patients, or those with arteriosclerosis.

Common chronic wounds include, but are not limited to pressure ulcers, venous ulcers, and the like. Pressure ulcers, also known as decubitus ulcers or bedsores, are localized injuries to the skin and/or underlying tissue usually over a bony prominence, as a result of pressure, or pressure in combination with shear and/or friction. Most commonly this will be the sacrum, coccyx, heels or the hips, but other sites such as the elbows, knees, ankles or the back of the cranium can be affected. The cause of pressure ulcers is pressure applied to soft tissue so that blood flow to the soft tissue is completely or partially obstructed. Shear is also a cause; shear pulls on blood vessels that feed the skin. Pressure ulcers most commonly develop in persons who are not moving about or are confined to wheelchairs. Pressure ulcers can be very difficult to prevent in critically ill patients, frail elders, wheelchair users (especially where spinal injury is involved) and terminally ill patients. Venous ulcers (stasis ulcers, varicose ulcers, or ulcus cruris) are wounds that are thought to occur due to improper functioning of venous valves, usually of the legs. The exact etiology of venous ulcers is not certain, but they are thought to arise when venous valves that exist to prevent backflow of blood do not function properly, causing the pressure in veins to increase. They are a major cause of chronic wounds, occurring in 70% to 90% of chronic wound cases. Venous ulcers develop mostly along the medial distal leg, and can be very painful.

### Bum Wounds

The instant invention also features the use of GDNF for treatment in cases where a subject suffers from burn wounds. Most burn wounds affect only the skin (epidermal tissue). Rarely, deeper tissues, such as muscle, bone, and blood vessels can also be injured. Burns are described according to the depth of injury to the dermis and are loosely classified into first (involving the epidermis), second (extending into superficial (papillary) dennis and/or extending into deep (reticular) dermis), third (extending through entire dermis), and fourth (extending through skin, subcutaneous tissue and into underlying muscle and bone) degrees. Burns are caused by a wide variety of substances and external sources such as exposure to chemicals, friction, electricity, radiation, and heat. Generally, the methods of the invention are suited to the treatment of first through third degree burns.

### Freezing Injury

The instant invention also features the use of GDNF for treatment in cases where a subject suffers from freezing injury, in particular, from wounds or tissue damage resulting from freezing injury. An exemplary freezing injury is frostbite. Frostbite is the medical condition in which localized damage is caused to skin and other tissues due to freezing. Frostbite is most common in body parts farthest from the heart and those with large exposed areas. Frostbite involves tissue destruction. Second-degree injury usually involves blisters (appearing 1-2 days after tissue becoming frozen.) The blisters may become hard and blackened, with time. Most of the injuries heal in one month, but the area may become permanently insensitive to both heat and cold. The GDNF treatment methodologies of the invention are suited for treatment of tissue damage, blisters, wounds and the like associated with frostbite and other freezing injuries.

### Diabetics

Diabetes mellitus is well known for its skin complications, usually leading to the formation of chronic debilitating ulcers (Levin, M. E. 1995, DiabetesCare 18, 1383-94; Cavanagh, P.R., et al. 1998, Ostomy Wound Manage 44, 6S-12S; Brem et al., 2003). Wound-healing impairment is characterized by the inability of the healing process to progress, thus leaving the wound susceptible to external infections as well as to deterioration of the underlying tissue, leading to morbidity and sometimes requires amputation (Brem, H. et al. Surg Technol Int 11, 161-7 (2003); Freedman, H., et al. Am J Surg 188, 31-5 (2004); Mousley, M. Nurs Times 99, 70-4 (2003); Wertheimer, E. Isr Med Assoc J 6, 287-9 (2004)). Accordingly, the instant invention also features the use of GDNF for treatment in cases where a subject suffers from ulcers associated with diabetes. In exemplary embodiments, the invention features use of GDNF for treatment of “diabetic foot ulcer” and/or “non-healing chronic diabetic ulcers.”

### Cosmetic Applications

Without being bound in theory, it is proposed that the GDNF compositions of the invention may have utility in the field of cosmetic applications, e.g., in dermatological application. For example, the GDNF compositions of the invention could be useful as. “antiaging” substances and/or may improve the appearance of wrinkles. It was noted in the wound healing experiments, described herein, that mice exhibited smoother skin following GDNF treatment. Other research on wound healing has produced much evidence showing the importance of peptides in improving the signs of aging (Lupo MP, Cole AL. Dermatol Ther. 2007; 20:343-349). Thus, in exemplary embodiments, the GDNF peptides of the invention can be used in methods to improve fine lines, skin texture, and/or hyperpigmentation in addition to their uses to influence wound healing. It is important for such application that the/protein peptide is stable in formula, deliverable to its target dermal site, and biologically active at this target site (Lupo M. Dermatol Surg. 2005; 31:832-836).

### Screening Assays

In other aspects, the invention features the use of GDNF peptides/proteins in screening assays, e.g., in screening assays for compounds that modulate one or more of the biological activities of GDNF in the processes of wound healing and/or hair growth. In one embodiment, the invention features contacting a composition comprising GDNF with a test compound and determining the ability of the test compound to upregulate, e.g., increase or enhance, the activity of GDNF (or a biologically active fragment thereof) such that a compound having the potential to increase hair growth or improve wound healing is identified. In another embodiment, the invention features contacting a cell expressing GNDF with a test compound and determining the ability of the test compound to upregulate, e.g., increase or enhance, the expression or activity of GDNF (or a biologically active fragment thereof) such that a compound having the potential to increase hair growth or improve wound healing is identified. In exemplary embodiments, the cell is a cell known in the art to play a role in wound healing and/or hair growth. A multitude of screening assay formats art known in the art and it is contemplated that screening assays of the invention can make use of labeled reagents, e.g., labeled GDNF proteins, unlabeled reagents, immobilized reagents, and the like. High throughput formats are also well known in the art and are contemplated as a preferred embodiment for the screening assays of the invention.

### VI. Pharmaceutical Compositions and Formulations

In other aspects, the invention features pharmaceutical formulations that include a therapeutically effective dose of isolated GDNF, or a biologically active fragment thereof, formulated in combination with at least one agent which facilitates administration of said GDNF, or a biologically active fragment thereof.

Topical formulations are often prepared in the form of emulsions. The term “emulsion,” as used herein refers to mixtures of two or more liquids, which may be in the form of a continuous phase and a disperse phase, for example. Exemplary emulsions may be in the form of creams, lotions, ointments, gels, etc. and may include, for example, oil-in-water emulsions, water-in-oil emulsions, multiple emulsions and microemulsions. These formulations will be prepared which contain from about 0.001 to 10 w/w % of the GDNF compositions of the present invention. These formulations will then be administered or applied to the desired areas, e.g., from 1 to 4 times daily. Alternatively, these formulations will be administered or applied to the desired areas less frequently, i.e., from 1 to 5 times a week. Formulations can be applied or administered, for example, every other day, every third day, and so forth. Administration or application may vary in frequency over the course of treatment.

The GDNF compositions may also be administered topically in the form of liposome delivery systems, such as small unilamellar vesicles, large unilamellar vesicles and multilamellar vesicles. Liposomes can be formed from a variety of phospholipids. such as cholesterol, stearylamine or phosphatidylcholines. A potential formulation for topical delivery of the hair treatment compositions used in the methods of the present invention utilizes liposomes such as described in U.S. Pat. Nos. 4,911,928 and 5,834,014.

Carriers for systemic administration include, for example, sugars, starches, cellulose and its derivatives, malt, gelatin, talc, calcium sulfate, vegetable oils, synthetic oils, polyols, alginic acid, phosphate buffer solutions, emulsifiers, isotonic saline and pyrogen-free water. Suitable carriers for parenteral administration include, for example, propylene glycol, ethyl oleate, pyrrolidone, ethanol, and sesame oil.

In exemplary embodiments, the GDNF compositions of the invention are formulated in gels or in nanoparticles. Exemplary gels include, for example, carboxymethylcellulose-based gels. Further exemplary gel formulations include, but are not limited to, polymeric gel formulations, in particular, those comprising a polymer selected from the group consisting of vinyl polymers, polyoxyethylene-polyoxy propylene copolymers, polysaccharides, proteins, poly (ethylene oxide), acrylamide polymers and derivatives or salts thereof. Such gel formulations are described in, e.g., U.S. Pat. No. 5,705,485; formulation with nanoparticles.

In other exemplary embodiments, the GDNF compositions of the invention are formulated in heparin, heparan sulphate (see e.g., US Application 2010/0056440), hyaluronic acid, lactic acid or in glycolic acid. In other exemplary embodiments, the GDNF compositions of the invention are formulated in combination with polymeric compounds (such as polylactic acid, polyglycolic acid, poly(lactide-co-glycolide) (PLGA) microparticles, etc.) or in liposomes. In yet other exemplary embodiments, the GDNF compositions of the invention are formulated in collagen-coated delivery systems or in combination with alginate, chitosan, lactide and/or lactide/glycolide copolymers. In yet other embodiments, the GDNF compositions of the invention are formulated as part of a topical dressing (e.g., within an adhesive bandage). In yet other embodiments, the GDNF compositions of the invention are formulated as slow release forms, in films (e.g., biodegradable or non-biodegradable firms. In yet other embodiments, the GDNF compositions of the invention are formulated in combination with PEG 400 or serum albumin (e.g., human serum albumin.)

In other exemplary embodiments, the GDNF compositions of the invention are formulated as hydrogel compositions, i.e., hydrogels or hydrogel formulations. In a preferred aspect of the invention, the hydrogels comprise GDNF. In a preferred aspect of the invention, the hydrogels comprise GDNF included within liposomes. Liposomes have been used in delivering bioactive compounds, for example, growth factors and/or cytokines, for therapeutics purposes due to low toxicity, lack of immune system activation and targeted delivery at the site of action.

In one aspect, the invention features a hydrogel composed of liposomes (e.g., liposomes including GDNF) and chitosan. Ogiso et al., have shown that the negatively charged liposomes diffuse to dermis and lower part of hair follicles, increasing the permeation of drug through the skin (Ogiso T. etal., (2001). J Drug Targeting.: 9. 49-59.) Chitosan, a natural polysaccharide polymer, has been used as a hydrogel mixed with liposomes to deliver growth factors at the injected site with slow release of the bioactive molecules. Earlier studies have shown that production of the vascular endothelial growth factor is up-regulated in wound healing when macrophages are activated by chitin/chitosan (review by Muzzarelli, see e.g., Muzzarelli, RAA. (2009) Carbohydrate Polymers: 76, 167-182). Moreover, Patel et al., have shown that GDNF-Chitosan blended nerve guides enhances both functional and sensory recovery in vivo (Patel M, et al., (2007). J tissue Eng. & Regenerative Med.: 1, 360-367). For further background, see e.g., Elcin YM et al. (1996) Artif. Cell Blood Substit. Immobil. Biotechnol.: 24, 257-271

Accordingly, in one aspect, the invention features a therapeutic delivery system, e.g., a drug delivery formulation, comprising liposomes containing GDNF, formulated into a hydrogel, e.g., chitosan, for administration in a therapeutic regimen described herein, for example, for administration to a wound site, e.g., in wound healing aspects of the invention. In an exemplary embodiment, the invention features hydrogels made according to the following process. Growth factor or cytokine, e.g. GDNF is loaded into liposomes and then mixed with hydrogel agent, e.g., chitosan. Briefly, liposomes are dissolved in appropriate solvent or buffer and into a thin film (e.g., air and/or gas dried.) Dries films are then resuspended and filteres through appropriately sized filters to generate small unilamellar vesicles. Liposome-encapsulated growth factor/cytokine, e.g. GDNF can be prepared by sonication of the lipids and growth factor/cytokine in appropriate weight/weight ration. To generate hydrogels, liposome-encapsulated growth factor/cytokine, e.g, GDNF can be added to a solution, e.g., a prechilled solution) of hydrogel polymer, for example, chitosan (dissolved in appropriate solvent/buffer) by gentle stirring for an appropriate time before applying at the site of administration, e.g., at the wound site.

It will be recognized by the skilled artisan that the compositions so-formulated can also include inactive ingredients, for example, preservatives, stabilizers, solubilizers, and the like: sodium chloride, sodium acetate trihydrate, glacial acetic acid, water for injection, and methylparaben, propylparaben, and m-cresol as preservatives and l-lysine hydrochloride as a stabilizer.

Exemplary inactive ingredients include, for example, buffer, for example, pH buffer e.g., sodium phosphate, potassium phosphate, histidine, or Tris-HCl, e.g., at a concentration of 10-50 mM; salt (for the purpose of tonicity modifier and solubilizer), e.g., NaCl and CaCl₂ at a concentration of 10-100 mM: sugar (for the purpose of protein-stabilizer, bulking agent, etc), e.g., sucrose or trehalose at a concentration of 10-100 mg/mL; polvol (for the purpose of tonicity modifier and bulking agent), e.g., mannitol or sorbitol at a concentration of 10-100 mg/mL; amino acid (for the purpose of tonicity modifier, bulking agent and stabilizer), e.g., glycine or arginine at a concentration of 10-100 mg/mL; polymer (for the purpose of bulking agent etc.), e.g., hydroxyethyl starch at a concentration of 10-50 mg/mL; surfactant (for the purpose of solubilizer, stabilizer and aggregation inhibitor), e.g., Tween-80, Tween-40, and/or SDS at a concentration of <1 mg/ml; preservative (for the purpose of antimicrobial preservation), e.g., benzyl alcohol or phenol e.g., at a concentration of 1-10 mg/ml; and/or antioxidant (for the purpose of antioxidant), e.g., ascorbic acid e.g., at a concentration of 1-10 mg/ml; and or other agents (with a purpose of protein-specific stabilization). Exemplary formulations are also described, for example, in U.S. Pat. No 8,383,114.

Administration may be by periodic injections of a bolus of the pharmaceutical composition or may be made more continuous by intravenous or intraperitoneal administration from a reservoir which is external (e.g., an IV bag) or internal (e.g., a bioerodible implant). See, e.g., U.S. Pat. Nos. 4,407,957, 5,798,113, and 5,800,828, each incorporated herein by reference.

Examples of parenteral delivery systems include, but are not limited to, ethylene-vinyl acetate copolymer particles, osmotic pumps, implantable infusion systems, pump delivery, encapsulated cell delivery, liposomal delivery, needle-delivered injection, needle-less injection, nebulizer, aeorosolizer, electroporation, hydrogels and transdermal patches. In some embodiments, compositions of the invention may be provided in lyophilized form as a dried powder or a cake.

In the case of injections of the GDNF compositions, GDNF proteins can be formulated in sterile physiological saline solution (e.g., 10 nM citrate and 150 mM sodium chloride), optionally including heparin, heparan sulphate, and/or glycerol.

In some embodiments, GDNF can be produced in Escherichia coli cells that contain an expression plasmid with a DNA insert encoding mature human GDNF. In such embodiments, it is preferred to engineer into the protein an N-terminal methionine.

The following examples illustrate the preparation of certain specific compounds according to the present technology. A skilled artisan appreciates that the invention is not limited to the exemplary work described or to the specific details set forth in the examples.

A skilled artisan further appreciates that the experimental conditions depicted in the following examples can be varied by as much as 2%, 5%, 10% or 20% above or below the listed amount, temperature, concentration, pH, time and rpm in order to optimize the conditions to achieve the desired results from the experiments.

### EXAMPLES

### Example 1: Transgenic Overexpression of GDNF

In a transgenic mouse model generated in the laboratory for a different hypothesis, when Gdnf is over-expressed under the Cathespin L promoter, it affects the hair follicle growth in mice. Further analysis suggested that the effect of GDNF on the hair follicles in these mice could be due to expression of Gdnf in cells expressing endogenous Cathespin L gene, cells of the outer and inner root sheath and is essential for regular hair follicle morphogenesis and cycling. Though we do not understand the mechanism how GDNF regulates hair follicle growth but Gdnf is expressed in embryonic skin where Gdnf mRNA is detected in both epithelial and mesenchymal components (Hellmich et al. 1996, Mech Dev 54:95-105).

Transgenic mice were generated by microinjection a DNA construct comprising the Cathespin L promoter operably linked to the Gdnf cDNA Ctsl promoter driven Gdnf transgene contains a 3 kb genomic fragment upstream of the rat Ctsl translational start site. The numbering is relative to the Ctsl transcriptional start site, designated by +1. The coding sequence of Gdnf-Gfp fusion gene was cloned downstream of the promoter, using standard methods as described in Manipulating the Mouse Embryo: A Laboratory Manual, 3rd edition, Cold Spring Harbor Laboratory Press; 2002, ISBN-10: 0879695919. The Cathespin L promoter is described in Charron et al. 2003, Biol Reprod, 8 1(3), 1641-1648. When we analyzed the FVB transgenic mice Tg(Ctsl-Gdnf) we detected ruffled fur by 3 wk of age. We prepared paraffin skin sections stained with H&E. Analysis of such revealed an increased number of hair follicles adjacent to normal hair development compared to skin section from control littermate. This phenotype was reproducible in a different genetic background, using the mouse strain B6C3H. The transgenic mice expressing mouse Gdnf under Cathespin L promoter (Tg-Ctsl-Gdnf) were generated in the FVBN/J and B6C3HF1 strain.

### Example 2: Accelerated Wound Healing of a Full-Thickness Wound

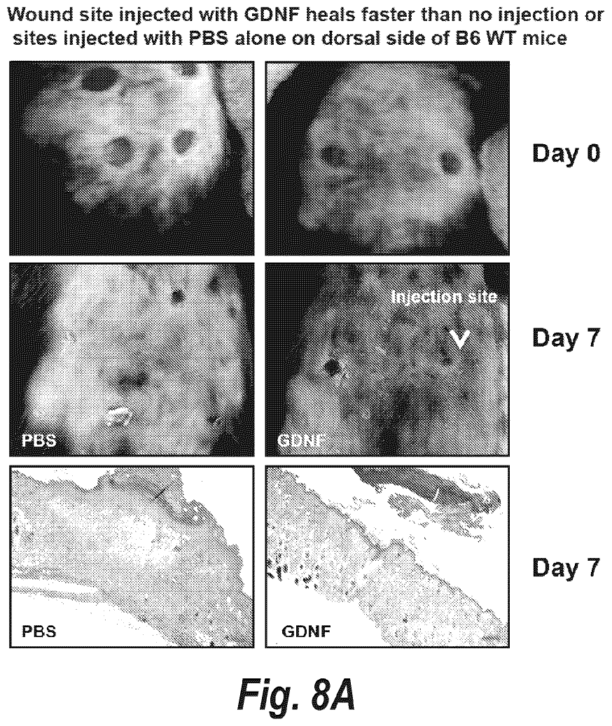

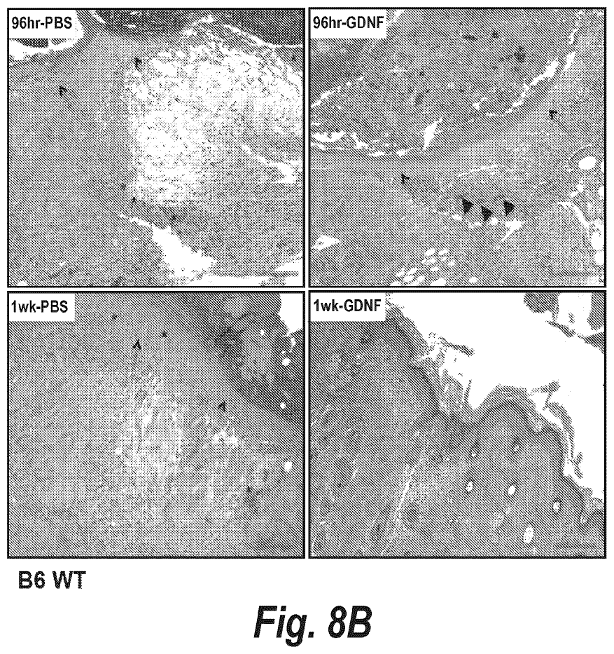

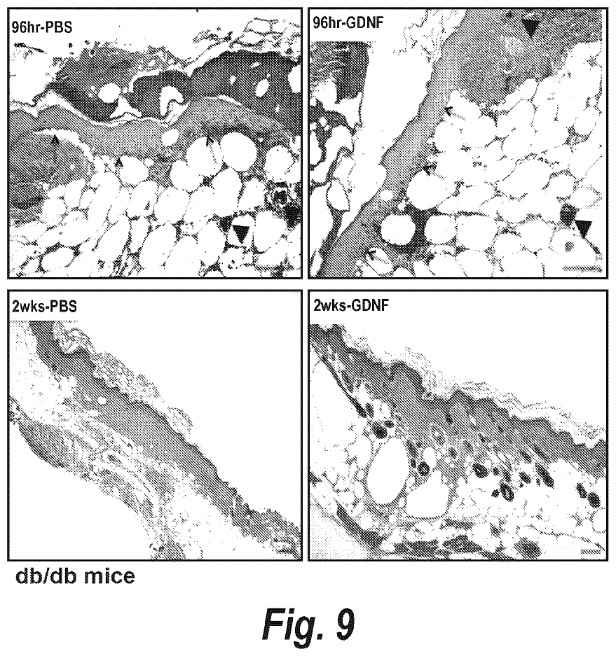

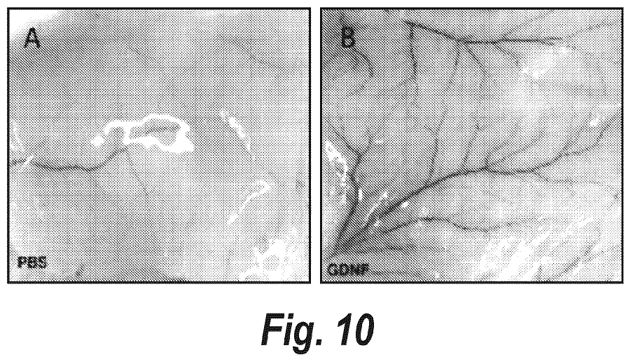

Wild type C57BL/6J (B6) or BKS. Cg-Dock7^{m}+/+ Lepr^{db} mice were anesthetized using isoflurane. Before wound setting, the hair was removed on the dorsal side using a clipper. Loose hair was removed with dry gauze dampened with 70% ethanol. Four 3 mm full skin wounds were made using a 3 mm biopsy punch needle. 100 μ† of PBS or rat GDNF recombinant protein (Ser78-Ile21 1) from R&D system (cat no. 512-GF) at concentrations of 0.1 mg/ml or 0.5 mg/ml diluted in PBS was injected either at the wound site or subcutaneously in the middle of the 4 wounds using 30 G needles, approximately 5-6 mm adjacent to the wounds. The mice were housed individually and monitored daily after surgery. Wound re-epithelialization and hair follicle development was determined by histology at 96 hrs, 6, 8, and 15 days after injection of the recombinant GDNF protein (FIGS. 8A, 8B and 9).

### Example 3: Dose Comparisons for Hair Follicles

For the GDNF injections we tested one dose of 100 μ† 0.5 mg/ml GDNF protein (R&D system, cat no. 512-GF) versus five 100 μ† injections of 0.1 mg/ml GDNF on alternate days.

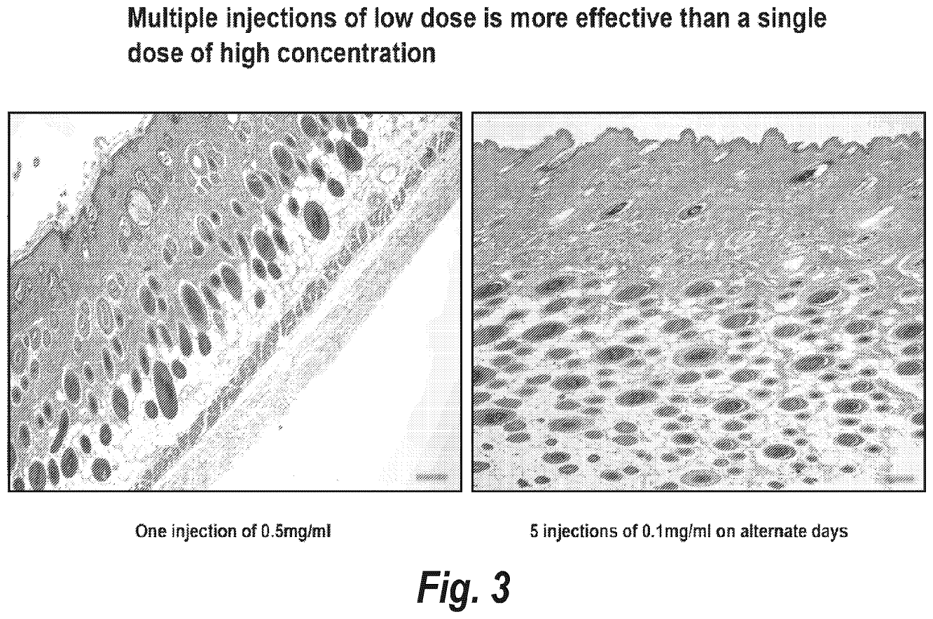

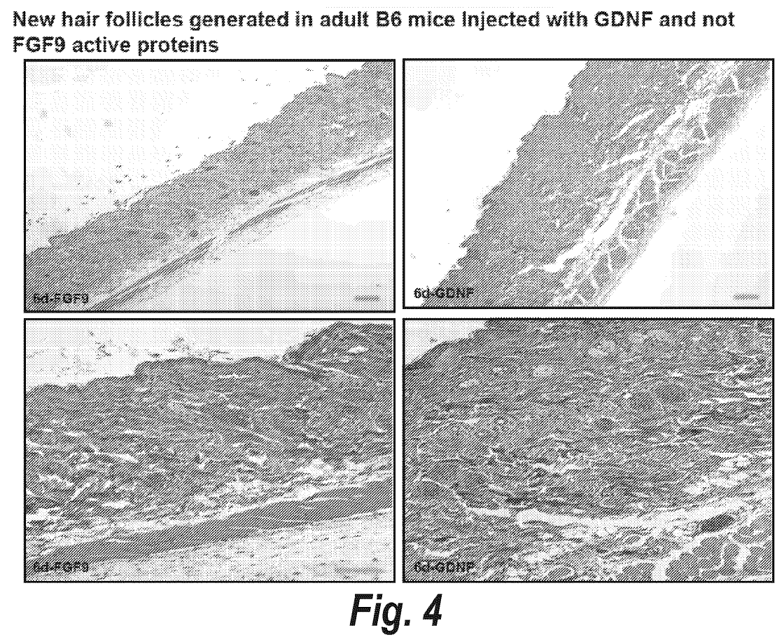

Skin tissue was harvested 15 days after injection (day 15) and paraffin sections were prepared and stained with H&E. In both groups an increase of hair follicles was detected, but a more striking increase when GDNF was administered at the lower dose over five time points, with the hair follicles being in different developmental stages (FIG. 3).

### Example 4: Dose Comparison for Hair Follicle Development

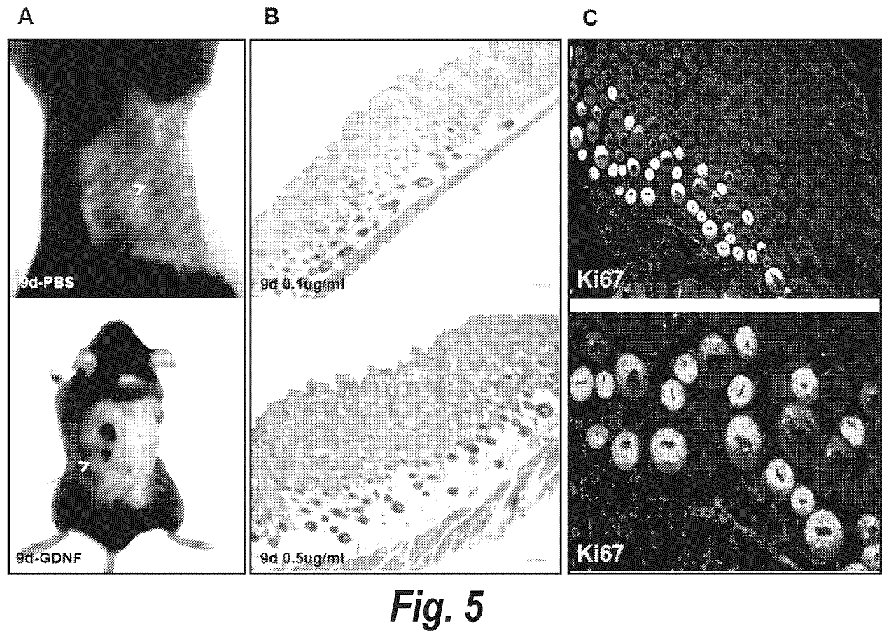

Adult C57BL/6 (B6) wild type mice were injected with 100 μ† of 0.1 mg/ml or 0.5 mg/ml recombinant GDNF protein (R&D system, cat no. 512-GF). The skin was analyzed after 9 days. One injection of 0.1 mg/ml was sufficient to induce hair follicle development at the injection site by day 9 (FIG. 5A). With the higher dose of 0.5 mg/ml more hair follicles were detectable (FIG. 5B). The rate of proliferation was assayed by 5-Bromo-2′-deoxyuridine (BrdU) labeling (Sigma, cat. no. B5002) according the protocols described by Sanjay et al. 2008, Methods in Mol Biol 438, 335-343. For this BrdU was injected intraperitoneally (i.p.) one day before collecting the skin. Most of the new hair follicles in the GDNF group stained positively indicating that these are proliferating while (data not shown). Alternatively skin sectioned were immunostained with Ki67 antibody, as Ki67 protein is an established marker for cell proliferation. Ki67 is expressed during active phase of the cell cycle G1, S and G2 and absent from resting cells GO. The staining of hair follicle cells with Ki67 antibody shows that they are in proliferative stage on day 9 after GDNF injection (FIG. 5C).

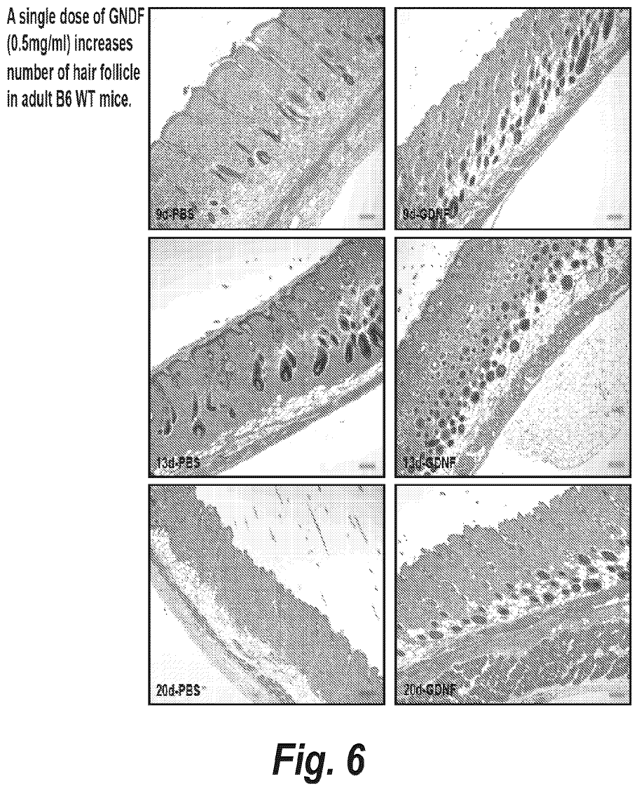

Adult B6 mice were injected with 100 μ† of PBS or 0.5 mg/ml of GDNF (all injections were on the dorsal side and subcutaneous (s.c.)) and skin was isolated at day 6, 9, 13, 15, 20, and 30 after injections, paraffin embedded and sectioned for histological analysis. A dramatic increase in hair follicle numbers was observed on day 9 and 13 compared to mice injected with PBS (FIG. 6). By day 20 hair regeneration is complete in mice injected with PBS. Surprisingly, hair follicles are still proliferating in the GDNF injected group (FIG. 6).

### Example 5: GDNF Signaling in the Epidermis

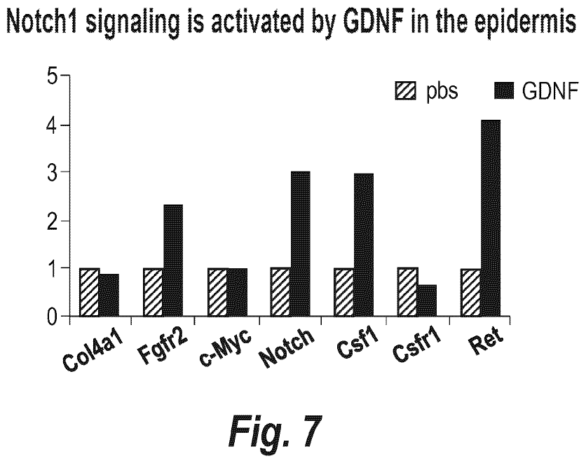

We analyzed the effects of GDNF (R&D system, cat no. 512-GF) on skin by quantitative RT-PCR (q-RT-PCR). For this total RNA was extracted from about 100 mg, about 8 mm diameter, skin tissue isolated from area injected either with PBS or 0.5 mg/ml GDNF protein 6 days after injection. RNA isolation was performed using Trizol Plus kit from Invitrogen. qRT-PCR of specific genes listed below was performed using One step Real-time RT-PCR SYBR green kit from Applied biosystems. Beta actin was used as internal control. Transcripts of selected genes were analyzed and normalized to beta-actin. The PCR protocol used here followed the RT-PCR protocol according to Applied Biosystems One step Real-time RT-PCR protocols. We tested expression of coHal, Ret, Fgfrl, c-myc, Notchl, Csfl and Csfrl. A four-fold increase in Ret transcript was seen, and a three-fold increase for Notchl and Csfl. For Ret and Notchl one a role in hair growth has been described previously (Kato et al. 2001; Vauclair et al. 2005), and it may be that the action of GDNF is mediated by upregulating these. To date, it has not been shown that Notchl is part of the GDNF signaling pathway, and this is the first time seeing this novel action of GDNF. We do not observe a change in c-myc, col4al and csfrl expression. For example c-myc is not described as part of the GDNF signaling pathway and can be considered as negative control. While, it is known that c-myc over-expression results in proliferation epidermal cells, it does not seem to play a role here (FIG. 7).

The primers used for the q-RT-PCR are:

Fgfr2-Forward
(SEQ ID NO: 14)
5′-ctctctacgtcatagttgaatatg-3
Fgfr2-Reverse
(SEQ ID NO: 15)
5′-atatccctggccaggccaaagtct-3′
Ret-Forward
(SEQ ID NO: 16)
5′-agatgtttatgaggaagattccta-3
Ret-Reverse
(SEQ ID NO: 17)
5′-Tcctcgctgcagttgtctggcctc-3′
Col4al-Forward
(SEQ ID NO: 18)
5′-atgccctttctcttctgcaa-3′
Col4al-Reverse
(SEQ ID NO: 27)
5′-ctgcggaatctgaatggtct-3′
Csfl-Forward
(SEQ ID NO: 19)
5′-gatccctgagtctgtcttccacct-3′
Csfl-Reverse
(SEQ ID NO: 20)
5′-cagttccacctgtctgtcctcatcc-3′
Csflr-Forward
(SEQ ID NO: 21)
5′-gtaaagtggatggccccagagagc-3
Csflr-Reverse
(SEQ ID NO: 22)
5′-taggctccaggtcccagcaggactg-3′
c-Myc-Forward
(SEQ ID NO: 23)
5′-cagctcgcccaaatcctgtacctcgt-3′
c-Myc-Reverse
(SEQ ID NO: 24)
5′-cagacaccacatcaatttcttcctc-3′
Notchl-Forward
(SEQ ID NO: 25)
5′-tgaagaacggagccaacaaggacatgc-3′
Notchl- Reverse
(SEQ ID NO: 26)
5′-gcaatcggtccatgtgatccgtgatgt-3′

### Example 6: GDNF Accelerates Wound Healing in B6 Mice

Mice were anesthetized using isoflurane and fur removed using clipper from the dorsal side. Loose fur was removed with dry gauze dampened with 70% ethanol. Equal size 3 mm of full thickness wounds were set using a biopsy punch needle on the dorsal side of adult B6 mice. The mice were housed individually and monitored daily after surgery. Wounds were photographed on day 0 before injection and after one week, day 0 is counted as day of injection. Only one wound site was injected once with 100 μ† of 0.5 mg/ml GDNF (R&D system, cat no. 512-GF) (arrow), the other wound was injected with PBS (vehicle control). The wound site that was injected with 100 μ† of 0.5 mg/ml GDNF healed faster than the wound sites injected with PBS. After one week mice were sacrificed and tissue around the original wound was isolated to prepare sections. The sections of wound sites injected with PBS or GDNF were stained using H&E. By one week all layers of skin, epidermis, dermis and hair follicles are present at the wound site injected with GDNF compared to PBS injected sites (FIG. 8A). In the PBS control the skin does not show the three layers, but is still in the reconstruction phase after one week. The wound repair starts as early as 48 hrs after the wound is set with immune cells detectable. At the wound site red blood cell (RBC) infiltration is seen in both groups, but to a higher extent in the GDNF group (data not shown). By 96 hrs a complete layer of epithelium was observed throughout the wound site only for the GDNF group (FIG. 8B arrow) and many new blood vessels (arrowheads) are detectable in the GDNF injected site. In the PBS group less immune cells and blood vessels are detectable compared to the GDNF group. At the one week time point no new hair follicles are detectable in the PBS group.

### Example 7: GDNF Accelerates Wound-Healing in Diabetic Mice

As diabetic model, we chose the diabetic mouse strain BKS.Cg-Dock7^{m}+/+ Lepr^{d}^{_{b}}/J (db/db: available from The Jackson Laboratory stock number 000642). Mice were anesthetized using isoflurane and fur removed using clipper from the dorsal side. Loose fur was removed with dry gauze dampened with 70% ethanol before 3 mm full thickness wounds were set into the dorsal skin creating 4/wounds per mouse using a biopsy punch needle. The mice were housed individually and monitored daily after surgery. We treated the wounds either with 100 μ† PBS or 100 μ† of 0.5 mg/ml GDNF recombinant protein (R&D system, cat no. 512-GF). Mice were sacrificed at several time points and tissues was collected, fixed in formalin, sectioned and sections were stained with H&E. 96 hours after wound setting and GDNF treatment, we detected neovascularization and blood vessel formation (FIG. 9A arrowhead) more pronounced in the GDNF group compared to the PBS group. The increase in new blood vessel formation in db/db mice treated with GDNF can be seen at the one week time point. Compared to wild type B6 mice wound healing in diabetes, takes two weeks instead of one week when injected with 0.5 mg/ml of GDNF but at a higher dose (250 μ† of 0.5 mg/ml GDNF or 2.5fold more). The healing process is greatly accelerated after GDNF treatment even in the db/db mice.

### Example 8: GDNF Induces Blood Vessel Formation

FVB/NJ mice (n=5) (available from The Jackson Laboratory, cat no. 001800) were s.c. injected with 100 μ† of PBS or 100 μ† of 0.5 mg/ml GDNF protein (R&D system, cat no. 512-GF). 9 days after the injection the mice were sacrificed and the skin was isolated, photographed and fixed in formalin for histology. When the skin was isolated it was very striking that that the skin was showed more blood vessels with more branching in the GDNF group (Fig. xx). Analyzing the H&E stained sections confirmed an increased number of blood vessels around the GDNF injection site in comparison to the PBS injected tissue.

### Example 9: Hydrogel Formulations for Therapeutic Admilistration

An optimized formulation of liposome-encapsulated GDNF in hydrogel is prepared for the growth factor to be applied at the wound site. The growth factor is loaded into the liposomes and mix with chitosan. Briefly, liposomes (from Sigma # L4395) are dissolved in 10 ml of chloroform:methanol mixture (2:1) in round bottom flask and air dried to thin film by jet stream of argon gas at 40° C. The thin film is then hydrated in water and passed through 0.2 um polycarbonate filters a few times to get small unilamellar vesicles. The liposome-encapsulated GDNF is prepared by sonication of the lipids and growth factor in 0.250:1 ratio (w/w) of growth factor to the lipid. These liposomes are added to a prechilled solution of chitosan (1.8% wt/vol in 2% acetic acid) by gentle stirring for 10 minutes before applying at the wound site.

The present invention and its embodiments have been described in detail. However, the scope of the present invention is not intended to be limited to the particular embodiments of any process, manufacture, composition of matter, compounds, means, methods, and/or steps described in the specification. Various modifications, substitutions, and variations can be made to the disclosed material without departing from the spirit and/or essential characteristics of the present invention. Accordingly, one of ordinary skill in the art will readily appreciate from the disclosure that later modifications, substitutions, and/or variations performing substantially the same function or achieving substantially the same result as embodiments described herein can be utilized according to such related embodiments of the present invention. Thus, the following claims are intended to encompass within their scope modifications, substitutions, and variations to processes, manufactures, compositions of matter, compounds, means, methods, and/or steps disclosed herein.

The contents of any patents, patent applications, and references cited throughout the specification are herein incorporated by reference in their entireties.

1-23. (canceled)

24. A method for promoting cutaneous wound healing, the method comprising:
administering to a cutaneous wound of a subject in need thereof a therapeutically effective amount of a pharmaceutical composition comprising recombinant human glial cell-derived neurotrophic factor (GDNF), wherein the therapeutically effective amount promotes healing of the cutaneous wound.

25. The method of claim 24, wherein the therapeutically effective amount promotes filling and epithelialization of the cutaneous wound.

26. The method of claim 24, wherein the therapeutically effective amount promotes reestablishment of a skin barrier on the cutaneous wound.

27. The method of claim 24, wherein the pharmaceutical composition is administered subcutaneously to the cutaneous wound.

28. The method of claim 24, wherein the pharmaceutical composition is administered topically to the cutaneous wound.

29. The method of claim 24, wherein the method comprises repeatedly administering the pharmaceutical composition to the cutaneous wound.

30. The method of claim 24, wherein the cutaneous wound is selected from an incision, a laceration, an abrasion, a puncture wound, a penetration wound, a surgical wound, an ulceration, a burn, a contusion, a hematoma, and a crush injury.

31. The method of claim 24, wherein the pharmaceutical composition further comprises an anti-inflammatory agent.

32. The method of claim 24, wherein the pharmaceutical composition further comprises an antibiotic.

33. A method for promoting vascularization of a wound, the method comprising:
administering to a wound of a subject in need thereof a therapeutically effective amount of a pharmaceutical composition comprising recombinant human glial cell-derived neurotrophic factor (GDNF), wherein the therapeutically effective amount promotes vascularization of the wound, and wherein the wound is selected from a chronic wound, a burn wound, and a freezing injury.

34. The method of claim 33, wherein the wound is a chronic wound.

35. The method of claim 34, wherein the chronic wound is selected from a pressure ulcer and a venous ulcer.

36. The method of claim 34, wherein the chronic wound is a diabetic ulcer.

37. The method of claim 33, wherein the wound is a burn wound.

38. The method of claim 33, wherein the wound is a freezing injury.

39. The method of claim 38, wherein the freezing injury is frostbite.

40. The method of claim 33, wherein the pharmaceutical composition is administered topically to the wound.

41. The method of claim 33, wherein the method comprises repeatedly administering the pharmaceutical composition to the wound.

42. The method of claim 33, wherein the pharmaceutical composition further comprises an anti-inflammatory agent.

43. The method of claim 33, wherein the pharmaceutical composition further comprises an antibiotic.
